(19) United States
(12) Patent Application Publication
(10) Pub. No.: US 2014/0155861 A1
(43) Pub. Date: Jun. 5, 2014

(54) EXTRUSION METHODS AND DEVICES FOR DRUG DELIVERY

(71) Applicant: Presage Biosciences, Inc., Seattle, WA (US)

(72) Inventors: Edward Gallagher, Bellevue, WA (US); Alicia Moreno-Gonzelez, Redmond, WA (US); Worm Lund, SR., Bellevue, WA (US); Worm Lund, JR., Bellingham, WA (US); Marc Grenley, Seattle, WA (US)

(73) Assignee: Presage Biosciences, Inc., Seattle, WA (US)

(21) Appl. No.: 13/962,868

(22) Filed: Aug. 8, 2013

Related U.S. Application Data
(60) Provisional application No. 61/815,674, filed on Apr. 24, 2013.
(60) Provisional application No. 61/680,847, filed on Aug. 8, 2012.

(51) Int. Cl. A61B 17/34; A61M 5/32

(52) U.S. Cl. CPC A61B 17/3403; A61M 5/3298
USPC 604/506; 604/173; 428/137

(57) ABSTRACT

Disclosed herein are devices and methods for delivering agents to a solid tissue.

This application claims priority under 35 USC §119(e) to U.S. Provisional Application 61/680,847 filed Aug. 8, 2012 and U.S. Provisional Application 61/815,674 filed Apr. 24, 2013, each of which are incorporated by reference in their entirety.

### TECHNICAL FIELD

In general, the disclosed embodiments relate to methods and devices for the introduction and subsequent evaluation of agents to solid tissue, and in particular to the simultaneous introduction of a plurality of agents to the tissue in vivo.

### BACKGROUND

The cost of new drug development from discovery through phase III trials is estimated to be between $800 million and $1.7 billion and the process can take between eight and ten years. Despite success in classical anticancer models, many cancer therapeutics do not reach the clinic. Numerous drug candidates fail during clinical trials. It is estimated that more than 90% of cancer-related therapeutics will fail phase I or II clinical trial evaluation. The failure rate in phase III trials is almost 50%.

There is a need in the art for improved methods and devices for delivering and evaluating cancer therapeutics. The present invention addresses these and needs, and offer other related advantages.

### SUMMARY OF THE INVENTION

In one aspect, the disclosure provides for a device, comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks. In some embodiments, the device further comprises at least one adjustable leg, wherein the at least one adjustable leg is attached to the bottom block. In some embodiments, there are four adjustable legs. In some embodiments, the at least one leg is vertically and horizontally adjustable. In some embodiments, the bottom block is stationary. In some embodiments, the top block moves vertically relative to the bottom block. In some embodiments, the top block moves along guide rods attached to the bottom block. In some embodiments, the device further comprises a system to control vertical movement of the top block. In some embodiments, the first and second plurality of holes are arranged in substantially parallel rows. In some embodiments, the device further comprises at least one needle. In some embodiments, a control attachment is attached to the at least one needle. In some embodiments, the control attachment stops the insertion of the at least one needle, thereby controlling depth of needle insertion into the solid tissue. In some embodiments, the device further comprises at least one spring, wherein the at least one spring is in substantial contact with the adjustable leg and the bottom block. In some embodiments, the device further comprises a guiding rod that penetrates the bottom block. In some embodiments, the guiding rod is inserted into a solid tissue. In some embodiments, the guiding rod positions the needles on a solid tissue.

In one aspect, the disclosure provides for a device comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks.

In one aspect, the disclosure provides for a method of operating a device comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks, comprising: inserting one or more needles through the top block and the bottom block into a solid tissue, wherein at least one needle has a control attachment, and simultaneously move the top block away from the bottom block and injecting at least one agent into the solid tissue. In some embodiments, the at least one needle is inserted into the solid tissue in vivo. In some embodiments, the solid tissue is a tumor. In some embodiments, the control attachment controls the depth of needle insertion. In some embodiments, the at least one agent is injected in a column. In some embodiments, the length of the column is from 1-10 millimeters. In some embodiments, the one or more needles comprises at least two needles. In some embodiments, the one or more needles comprises at least five needles.

In one aspect, the disclosure provides for a method of evaluating at least one agent in a solid tissue, comprising placing at least a portion of the solid tissue under the device comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks, inserting one or more needles through the top block and the bottom block into the solid tissue, wherein at least one needle has a control attachment, simultaneously move the top block away from the bottom block, injecting at least one agent into the solid tissue, and evaluating an effect of the at least one agent. In some embodiments, the solid tissue is from a human, a mouse or a rat. In some embodiments, the plurality of agents comprises an agent selected from a protein agent, a peptide agent, a polypeptide agent, a peptidomimetic agent, an antibody agent, a small molecule agent, a small interfering RNA-encoding polynucleotide, an antisense RNA-encoding polynucleotide, or a ribozyme-encoding polynucleotide. In some embodiments, the at least one agent comprises a plurality of agents. In some embodiments, the method is performed in an organism. In some embodiments, the at least one agent has been systemically delivered to the organism. In some embodiments, the plurality of agents comprises a chemotherapeutic agent. In some embodiments, the plurality of agents comprises a small molecule agent. In some embodiments, the plurality of agents comprises an agent that interferes with RNA activity. In some embodiments, the plurality of agents comprises an anticancer agent. In some embodiments, the plurality of agents further comprise at least one position marker. In some embodiments, the at least one position marker is selected from the group consisting of a fluorescent dye, a nanoparticle, a GCMS tag molecule, a positive control, and a negative control. In some embodiments, the at least one position comprise a fluorescent dye. In some embodiments, at least two agents are injected into a same location. In some embodiments, the rate of injecting at least one agent is at least 0.1 μl/min. In some embodiments, the rate of injecting at least one agent is in the range of about 0.4 to about 4 μl/min. In some embodiments, the rate of the movement of the top block is at least 0.1 mm/min. In some embodiments, the rate of the movement of the top block is in the range of about 0.5 to about 5 mm/min. In some embodiments, the solid tissue is a tumor. In some embodiments, the tumor comprises at least one cancer cell selected from the group consisting of a prostate cancer cell, a breast cancer cell, a colon cancer cell, a lung cancer cell, a brain cancer cell, and an ovarian cancer cell. In some embodiments, the tumor comprises a cancer selected from the group consisting of adenoma, adenocarcinoma, squamous cell carcinoma, basal cell carcinoma, small cell carcinoma, large cell undifferentiated carcinoma, chondrosarcoma and fibrosarcoma. In some embodiments, the plurality of agents is delivered to spatially defined locations within the solid tissue. In some embodiments, the plurality of agents is delivered along parallel axes within the solid tissue. In some embodiments, the plurality of agent is delivered to column-shaped regions along parallel axes within the solid tissue. In some embodiments, wherein the one or more needles comprises at least 2 needles. In some embodiments, wherein the one or more needles comprises at least 5 needles. In some embodiments, wherein the one or more needles comprises at least 10 needles. In some embodiments, the evaluation comprises detecting an altered physiological state of the solid tissue. In some embodiments, the evaluation comprises detecting the activity or toxicity of the at least one agents on the solid tissue. In some embodiments, the evaluation comprises imaging the solid tissue. In some embodiments, the imaging comprises radiographic imaging, magnetic resonance imaging, positron emission tomogoraphy, or biophotonic imaging. In some embodiments, the imaging occurs before, during, or after introduction of the plurality of the at least one agent. In some embodiments, the evaluation comprises histology sectioning. In some embodiments, the evaluation comprises determining an effect of at least two agents on a same position within the region of the solid tissue. In some embodiments, the evaluation comprises determining an effect of at least two agents on adjacent position within the region of the solid tissue.

In one aspect, the disclosure provides for a method of evaluating at least one agent in a solid tissue, comprising placing at least a portion of the solid tissue under the device comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks, inserting one or more needles through the top block and the bottom block into the solid tissue, wherein at least one needle has a control attachment, simultaneously move the top block away from the bottom block, injecting at least one agent into the solid tissue, and evaluating an effect of the at least one agent, wherein the one or more needles comprises at least 2 needles, wherein the needles are part of a needle array device.

In one aspect, the disclosure provides for a method of evaluating at least one agent in a solid tissue, comprising placing at least a portion of the solid tissue under the device comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks, inserting one or more needles through the top block and the bottom block into the solid tissue, wherein at least one needle has a control attachment, simultaneously move the top block away from the bottom block, injecting at least one agent into the solid tissue, and evaluating an effect of the at least one agent, wherein the one or more needles comprises at least 5 needles, wherein the needles are part of a needle array device.

In one aspect, the disclosure provides for a method of evaluating at least one agent in a solid tissue, comprising placing at least a portion of the solid tissue under the device comprising a top block having a first plurality of holes sized to allow a needle to pass through the top block and a bottom block having a second plurality of holes sized to allow a needle to pass through the bottom block, wherein the top and bottom blocks are in a substantially parallel arrangement and wherein the first and second plurality of holes are positioned so as to allow one or more needles to pass through a hole in the top block and the bottom block in a path substantially vertical to the plane of both blocks, inserting one or more needles through the top block and the bottom block into the solid tissue, wherein at least one needle has a control attachment, simultaneously move the top block away from the bottom block, injecting at least one agent into the solid tissue, and evaluating an effect of the at least one agent, wherein the one or more needles comprises at least 10 needles, wherein the needles are part of a needle array device.

In one aspect, the disclosure provides for a device for delivering an agent to a solid tissue, comprising: a plurality of needles, wherein one or more needles of said plurality of needles have a first end and a second end, a reservoir, wherein said reservoir is in fluid communication with said first end of said one or more needles, a plunger, wherein said plunger is coupled to said reservoir, and a controller, wherein said controller controls the rate of fluid delivery when said one or more needles are withdrawn from said solid tissue. In some embodiments, the device further comprises a central placement needle. In some embodiments, the placement needle determines the correct orientation of the device on the tumor. In some embodiments, the placement needle is in fluid communication with the reservoir. In some embodiments, the controller is coupled to said plunger. In some embodiments, the agent is delivered to said solid tissue with said plunger being locked. In some embodiments, the device further comprises a loading mechanism, wherein said loading mechanism is fluidly coupled to the second end of said one or more needles. In some embodiments, the loading mechanism comprises a Closed System Transfer Device (CSTD). In some embodiments, the device is handheld. In some embodiments, the device further comprises a depth-control mechanism. In some embodiments, the depth-control mechanism controls the depth of needle insertion. In some embodiments, the one or more needles comprises 2 or more needles. In some embodiments, the one or more needles comprises 5 or more needles. In some embodiments, the one or more needles comprises 10 or more needles. In some embodiments, the needles are arranged along parallel axes. In some embodiments, the one or more needles is an end-port needle. In some embodiments, the reservoir comprises 2 or more reservoirs. In some embodiments, the reservoir comprises 5 or more reservoirs. In some embodiments, the reservoir comprises 10 or more reservoirs. In some embodiments, the device of claim 62, wherein the one or more needles comprises 2 or more needles and the reservoir comprises 2 or more reservoirs, and wherein each of said 2 or more reservoirs is in fluid communication with a different one of said 2 or more needles. In some embodiments, each of the 2 or more reservoirs comprises a different agent. In some embodiments, the device further comprises a deairification mechanism. In some embodiments, the deairification mechanism removes at least a portion of air bubbles from said reservoir during loading of said agent. In some embodiments, the deairification comprises at least one compartment connected to said reservoir. In some embodiments, the at least one compartment provides venting and pressure equalization during loading of said agent. In some embodiments, the at least one compartment is sealed during agent delivery. In some embodiments, the device further comprises a guiding rod that penetrates the bottom block. In some embodiments, the guiding rod is inserted into the solid tissue. In some embodiments, the guiding rod positions the needles on the solid tissue.

In one aspect, the disclosure provides for a method of delivering an agent to a solid tissue of a subject, comprising inserting a plurality of needles into said solid tissue, and delivering said agent to said solid tissue, wherein rate of agent delivery is substantially controlled by the rate of needle withdrawal from said solid tissue. In some embodiments, the delivering is carried out with a device comprising: a plurality of needles, wherein one or more needles of said plurality of needles have a first end and a second end, a reservoir, wherein said reservoir is in fluid communication with said first end of said one or more needles, a plunger, wherein said plunger is coupled to said reservoir, and a controller, wherein said controller controls the rate of fluid delivery when said one or more needles are withdrawn from said solid tissue. In some embodiments, the delivering is carried out with a device comprising: a plurality of needles, wherein one or more needles of said plurality of needles have a first end and a second end, a reservoir, wherein said reservoir is in fluid communication with said first end of said one or more needles, a plunger, wherein said plunger is coupled to said reservoir, and a controller, wherein said controller controls the rate of fluid delivery when said one or more needles are withdrawn from said solid tissue, wherein the agent is delivered to said solid tissue with said plunger being locked. In some embodiments, the delivering is carried out with a device comprising: a plurality of needles, wherein one or more needles of said plurality of needles have a first end and a second end, a reservoir, wherein said reservoir is in fluid communication with said first end of said one or more needles, a plunger, wherein said plunger is coupled to said reservoir, and a controller, wherein said controller controls the rate of fluid delivery when said one or more needles are withdrawn from said solid tissue, wherein the device further comprises a deairification mechanism. In some embodiments, the agent is delivered during needle withdrawal from said solid tissue. In some embodiments, the agent is delivered only during needle withdrawal from said solid tissue. In some embodiments, the needle is part of a needle array device. In some embodiments, the needle array device comprises 2 or more needles. In some embodiments, the needle array device comprises 5 or more needles. In some embodiments, the needle array device comprises 10 or more needles. In some embodiments, the agent is delivered along an axis within said solid tissue. In some embodiments, the axis is one of a plurality of parallel axes. In some embodiments, the agent comprises an anti-cancer agent. In some embodiments, the anti-cancer agent is a small molecular agent. In some embodiments, the agent comprises a position marker. In some embodiments, the agent comprises a negative control. In some embodiments, the agent comprises a positive control. In some embodiments, the delivering comprises delivering two agents to said solid tissue. In some embodiments, two agents are delivered to a same region within said solid tissue. In some embodiments, the two agents are delivered to different regions within said solid tissue. In some embodiments, the agent is present in said solid tissue at a therapeutically effective concentration. In some embodiments, outside said solid tissue said agent is present at a concentration that is less than about 75% of the therapeutically effective concentration. In some embodiments, at least one agent is present in said solid tissue at a therapeutically effective concentration, and outside said solid tissue said agent is present at a concentration that is less than about 90% of the therapeutically effective concentration. In some embodiments, the agent is undetectable outside said solid tissue. In some embodiments, the solid tissue comprises a tumor. In some embodiments, the tumor is selected from the group consisting of a benign tumor and a malignant tumor. In some embodiments, the tumor is selected from the group consisting of a primary tumor, an invasive tumor and a metastatic tumor. In some embodiments, the tumor comprises at least one cancer cell selected from the group consisting of a prostate cancer cell, a lymph node cell, a breast cancer cell, a colon cancer cell, a lung cancer cell, a brain cancer cell, a melanoma cell, a sarcoma cell, and an ovarian cancer cell, or any combination thereof. In some embodiments, the tumor comprises at least one cancer cell selected from the group consisting of a lymphoma cell, a breast cancer cell, a melanoma cell, and a sarcoma cell, or any combination thereof. In some embodiments, the tumor comprises a cancer selected from the group consisting of adenoma, adenocarcinoma, squamous cell carcinoma, basal cell carcinoma, small cell carcinoma, large cell undifferentiated carcinoma, chondrosarcoma, lymphoma, sarcoma, and fibrosarcoma. In some embodiments, the solid tissue is selected from the group consisting of brain, liver, lung, kidney, prostate, ovary, spleen, lymph node, thyroid, pancreas, heart, skeletal muscle, intestine, larynx, esophagus, skin and stomach. In some embodiments, the method further comprises evaluating an effect of said agent on said solid tissue. In some embodiments, the evaluating is performed in vitro. In some embodiments, the evaluating is performed in vivo. In some embodiments, the evaluating comprises histology sectioning. In some embodiments, the evaluating comprises imaging said solid tissue. In some embodiments, the imaging comprises radiographic imaging, magnetic resonance imaging, positron emission tomogoraphy, or biophotonic imaging. In some embodiments, the imaging occurs during, or after introduction of said agents. In some embodiments, the evaluating comprises collecting and analyzing at least one biomarker for tumor cell death, cell signal changes, or proliferation/mitotic changes. In some embodiments, the evaluating comprises detecting an effect of said one or more agents on a proliferative gradient or multiple microenvironments of said solid tissue. In some embodiments, the evaluating comprises detecting at least one biomarker using mass spectrometry. In some embodiments, the mass spectrometry is imaging mass spectrometry. In some embodiments, the position marker comprises infrared dyes. In some embodiments, the position marker comprises fluorescent inks. In some embodiments, the positional marker comprises new methylene blue, isosulfan blue or rhodamina WT.

In one aspect, the disclosure provides for a method of delivering an agent to a solid tissue of a subject, comprising inserting a plurality of needles into said solid tissue; and delivering said agent to said solid tissue, wherein rate of agent delivery is substantially controlled by the rate of needle withdrawal from said solid tissue, wherein said needle is part of a needle array device, wherein said needle array device comprises 2 or more needles, wherein each of said needles comprises a different agent.

In one aspect, the disclosure provides for a method of delivering an agent to a solid tissue of a subject, comprising inserting a plurality of needles into said solid tissue; and delivering said agent to said solid tissue, wherein rate of agent delivery is substantially controlled by the rate of needle withdrawal from said solid tissue, wherein said needle is part of a needle array device, wherein said needle array device comprises 5 or more needles, wherein each of said needles comprises a different agent.

In one aspect, the disclosure provides for a method of delivering an agent to a solid tissue of a subject, comprising inserting a plurality of needles into said solid tissue; and delivering said agent to said solid tissue, wherein rate of agent delivery is substantially controlled by the rate of needle withdrawal from said solid tissue, wherein said needle is part of a needle array device, wherein said needle array device comprises 10 or more needles, wherein each of said needles comprises a different agent.

In one aspect, the disclosure provides for a method of delivering an agent to a solid tissue of a subject, comprising inserting a plurality of needles into said solid tissue; and delivering said agent to said solid tissue, wherein rate of agent delivery is substantially controlled by the rate of needle withdrawal from said solid tissue, wherein said needle is part of a needle array device, wherein said needle array device comprises 2 or more needles, wherein at least two of said needles comprise a same agent at different concentration.

In one aspect, the disclosure provides for a method of delivering an agent to a solid tissue of a subject, comprising inserting a plurality of needles into said solid tissue; and delivering said agent to said solid tissue, wherein rate of agent delivery is substantially controlled by the rate of needle withdrawal from said solid tissue, wherein said needle is part of a needle array device, wherein said needle array device comprises 5 or more needles, wherein at least two of said needles comprise a same agent at different concentration.

In one aspect, the disclosure provides for a method of delivering an agent to a solid tissue of a subject, comprising inserting a plurality of needles into said solid tissue; and delivering said agent to said solid tissue, wherein rate of agent delivery is substantially controlled by the rate of needle withdrawal from said solid tissue, wherein said needle is part of a needle array device, wherein said needle array device comprises 10 or more needles, wherein at least two of said needles comprise a same agent at different concentration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 shows fluorescent microscopy images of several inefficient experiments.

FIG. 2 shows potential sources of platform variability.

FIG. 3 shows an illustration of an exemplary device embodying principles of the present disclosure.

FIG. 4 shows an example of a platform for tumor stabilization using springs embodying principles of the present disclosure.

FIG. 5 shows a top view of needles with a control attachment embodying principles of the present disclosure.

FIG. 6 shows an exemplary device with a system for controlling vertical movement of a top block embodying principles of the present disclosure.

FIG. 7 shows an exemplary needle array device.

FIG. 8 shows an exemplary system and method of delivering agents to needles.

FIG. 9 shows results evaluating different injection methods with simplified experimental systems.

FIG. 10 depicts fluorescent microscopy images of three different injection methods.

FIG. 11 shows results from standard injection method and extrusion method with respect to efficiency, intratumor signal uniformity and column length.

FIG. 12 shows average number of positive regions per section of three different injection methods.

FIG. 13 shows average variance within section of three different injection methods.

FIG. 14 depicts a schematic of a hand held device embodying principles of the present disclosure.

FIG. 15 illustrates the injection of a solid tumor using a drug delivery device.

FIG. 16 illustrates a transfer assembly for loading one or more agents into the drug delivery device depicted in embodying principles of the present invention.

FIG. 17 depicts loading of an agent into a pressure chamber.

FIG. 18 illustrates an exemplary embodiment of the disclosure.

FIG. 19 depicts a transfer vessel of the device.

### DETAILED DESCRIPTION

General Overview

A device of the present disclosure can be used to screen therapeutic agents in one or more tissues. A device can be comprised of a plurality of needles. A device of the present disclosure can come in a variety of configurations.

The present disclosure provides for a device for delivery of at least one agent to a solid tissue, comprising one bottom block and one top block in a substantially parallel arrangement, each having a plurality of holes. The plurality of holes in the bottom and top block may guide the insertion of needles. In some cases, the size of holes may be controlled to allow needles of a certain size to pass through. The device may lead to improved accuracy of needle insertion and exquisite control of delivery of the at least one agent to a solid tissue. In some embodiments, the configuration can be comprised of two blocks wherein tissue is placed between the blocks before injection with a plurality of needles.

In some embodiments, the configuration is a handheld device wherein a user can inject therapeutic agents into a tissue.

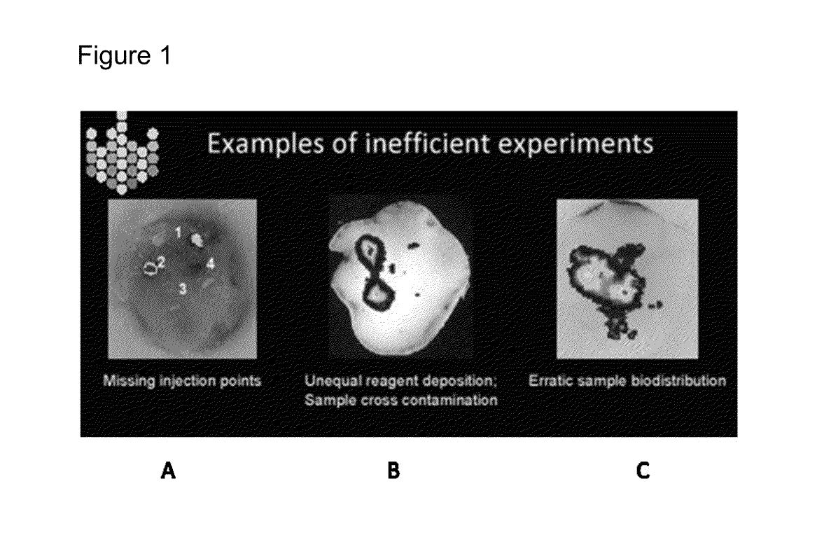

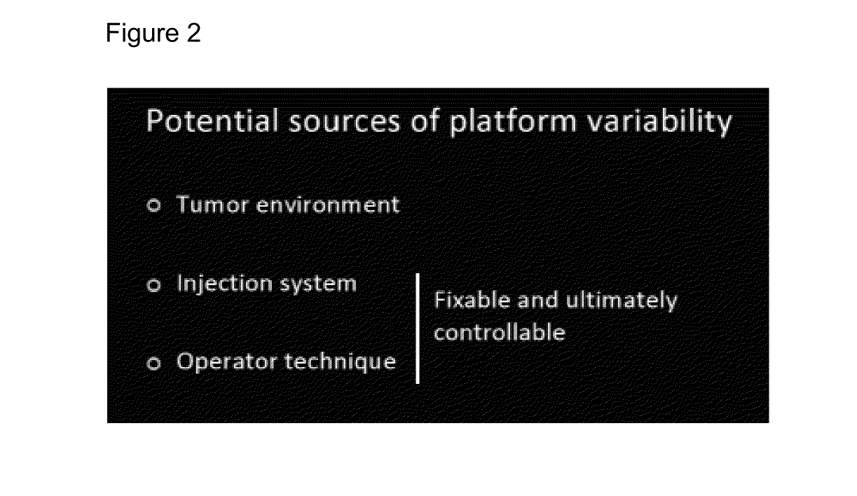

A device of the present disclosure may reduce potential sources of platform variability. For example, FIG. 1 shows fluorescent microscopy images of several experiments. FIG. 1A shows the fluorescent microscopy image of an experiment with missing injection points (there is no injection at points 3 and 4). FIG. 1B shows a fluorescent microscopy image of an experiment with unequal reagent deposition. Ultimately, the excess reagent deposited can lead to cross contamination. FIG. 1C shows a fluorescent microscopy image of an experiment with erratic sample biodistribution.

Without being limited by any theoretical explanation, potential sources of platform variability can include: (a) tumor environment; (b) injection system; (c) operator technique. The methods of this disclosure aim to reduce the variability the injection system and/or the operator technique. Improvement on these two aspects can lead to an improved method (e.g. improved precision and narrow biodistribution, of delivering at least one agent).

The examples and devices described herein are meant to be illustrative and not to limit scope of the present invention.

Block Devices

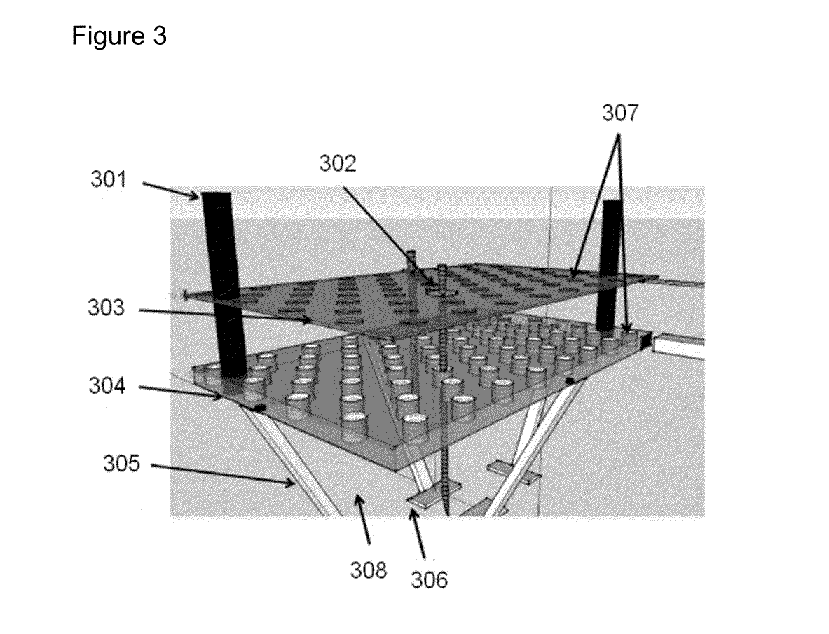

FIG. 3 depicts one type of device embodying principles of the present invention. The device assembly may comprise: a guiding rod 301, a needle with control attachment 302, a top block 303, a bottom block 304, a platform 308, a leg 305 with feet 305 and holes 307 in the top and the bottom block. The top block 303 and the bottom block 304 may be in a substantially parallel arrangement. The top block 303 and the bottom block 304 may be made of one or more materials. The top block 303 and the bottom block 304 may be made of different materials. The block material can be solid. Solid materials that can be used to make a top or a bottom block can include, but are not limited to: metals, plastics, glass, or any combination thereof. The material of the top and/or bottom block may be transparent.

The material of the top and/or bottom block may have a range of thickness. The block may be approximately: 0.5 mm, 1 mm, 2 mm, 3 mm, 4 mm, 5 mm, 6 mm, 7 mm, 8 mm, 9 mm, 1 cm, 1.2 cm, 1.5 cm, 1.75 cm, or 2 cm thick. In some embodiments, the block is less than 0.5 mm thick. In some embodiments, the block is more than 2 cm thick. The top block 303 can be of different thickness than the bottom block 304. The top block 303 and the bottom block 304 can be of the same thickness.

Each block may have a plurality of holes 307. The holes in each block 307 may have a variety of arrangements. In some embodiments the holes 307 within each block form substantially parallel rows. In other embodiments, the holes 307 may be arranged in other configurations. The number of holes in the block may correspond to the number of needles to be inserted. The number of hole(s) in each block may be 1, 2, 3, 4, 5, 6, 7, 8, 9, 10, 11, 12, 13, 14, 15, 16, 17, 8, 19, 20, 21, 22, 23, 24, 25, 26, 27, 28, 29, 30, 35, 40, 50, 60, 70, 80, 90, 100, 150, 200, 300, 400, or 500. In some embodiments, the number of holes can be over 1000 or over 2000.

The size of the holes in the blocks may vary. The size of the holes in the blocks may be of a size to accommodate a needle to pass through. Some non limiting examples can include: a hole greater than 4.572 mm in diameter to accommodate a gauge 7 needle; a hole greater than 4.191 mm in diameter to accommodate a gauge 8 needle; a hole greater than 3.759 mm in diameter to accommodate a gauge 9 needle; a hole greater than 3.404 mm in diameter to accommodate a gauge 10 needle; a hole greater than 3.048 mm in diameter to accommodate a gauge 11 needle; a hole greater than 2.769 mm in diameter to accommodate a gauge 12 needle; a hole greater than 2.413 mm in diameter to accommodate a gauge 13 needle; a hole greater than 2.108 mm in diameter to accommodate a gauge 14 needle; a hole greater than 1.829 mm in diameter to accommodate a gauge 15 needle; a hole greater than 1.473 mm in diameter to accommodate a gauge 17 needle; a hole greater than 1.270 mm in diameter to accommodate a gauge 18 needle; a hole greater than 1.067 mm in diameter to accommodate a gauge 19 needle; a hole greater than 0.9081 mm in diameter to accommodate a gauge 20 needle; a hole greater than 0.8192 mm in diameter to accommodate a gauge 21 needle; a hole greater than 0.7176 mm in diameter to accommodate a gauge 22 needle; a hole greater than 0.7176 mm in diameter to accommodate a gauge 22s needle; a hole greater than 0.6414 mm in diameter to accommodate a gauge 23 needle; a hole greater than 0.5652 mm in diameter to accommodate a gauge 24 needle; a hole greater than 0.5144 mm in diameter to accommodate a gauge 25 needle; a hole greater than 0.4636 mm in diameter to accommodate a gauge 26 needle; a hole greater than 0.4737 mm in diameter to accommodate a gauge 26s needle; a hole greater than 0.4128 mm in diameter to accommodate a gauge 27 needle; a hole greater than 0.3620 mm in diameter to accommodate a gauge 28 needle; a hole greater than 0.3366 mm in diameter to accommodate a gauge 29 needle; a hole greater than 0.3112 mm in diameter to accommodate a gauge 30 needle; a hole greater than 0.2604 mm in diameter to accommodate a gauge 31 needle; a hole greater than 0.2350 mm in diameter to accommodate a gauge 32 needle; a hole greater than 0.2096 mm in diameter to accommodate a gauge 33 needle; a hole greater than 0.1842 mm in diameter to accommodate a gauge 34 needle.

In some embodiments, the device can be configured to be used with any and/or all standard-sized needles. The size of holes 307 can be engineered to allow needles of a specific gauge to pass through them. The size of the holes 307 can be independently controlled to allow needles of specific gauge to pass through them. Generally, holes 307 in the top block 303 and the bottom block 304 may be aligned in such a way that the insertion trajectory of needles can be substantially perpendicular to the plane of the blocks. In some cases, two holes 307, one in the top block 303 and one in the bottom block 304, defining the insertion trajectory may be the same size. The size of holes 307 within a block may be uniform or different. In some embodiments, when the size of the holes is substantially uniform, needles of the same size can be used. In some embodiments, when the size of the holes is different, needles of different sizes can be used.

Guiding rods may be used to guide the movement of a block. There may be 1, 2, 3, 4, 5, 6, 7, 8, 9, 10 or more guiding rods. FIG. 3 depicts an exemplary embodiment wherein a guiding rod 301 may be used to guide the movement of the top block 303. Guiding rods may be attached to the bottom block 304. Guiding rods 301 may be permanently attached to the bottom block 304. The guiding rods 301 may penetrate the bottom block 304 and be inserted in the solid tissue and be used as a guide for subsequent positioning of the needles 302. The guiding rods 301 can control the movement trajectory of the top block 303. The guiding rods 301 may be substantially perpendicular to the bottom block 304 and/or the top block 303. Guiding rods may be substantially parallel to each other. In some cases, the top block 303 can move closer to and further from the bottom block 304 (e.g. vertically). Guiding rods 301 can be permanently attached to a block, as depicted in FIG. 3.

In other embodiments, guiding rods may not be permanently attached to a block. For example, without being limiting, the rods can be attached to the bottom block via clamps, which may be permanently attached to the side of the bottom block, allowing for disassembly of the device.

Blocks of the device can be placed on platforms. Platforms can be adjustable. Platforms can provide support for the block. In some embodiments, platforms can be optional. Platforms can have a variety of shapes or configurations. FIG. 3 depicts an exemplary embodiment wherein a platform is supporting a bottom block. The platform 306 can be underneath the bottom block 304. The platform 306 may provide support for the stationary bottom block 304. In FIG. 3, the platform is comprised of 4 legs 305, each attached to one side of the bottom block 304. However, other leg configurations with 1, 2, 3, 5, 6, 7, 8, 9, 10, 11, 12 or more legs is contemplated.

The other end of the leg 305 can be attached to a supporting surface 308. The legs can be of different shapes (e.g. cylindrical, rectangular or square). The legs can be of any shape. The legs can be vertically adjustable. The legs can be horizontally adjustable. The legs can be horizontally and vertically adjustable. After placing a solid tissue or a subject in the device, the adjustable platform can allow substantially improved tissue and/or subject stabilization during inserting of needles and injection of at least one agent. Without being bound to a particular theory, an adjustable platform can result in greater insertion precision, narrower biodistribution, and/or less sample cross contamination.

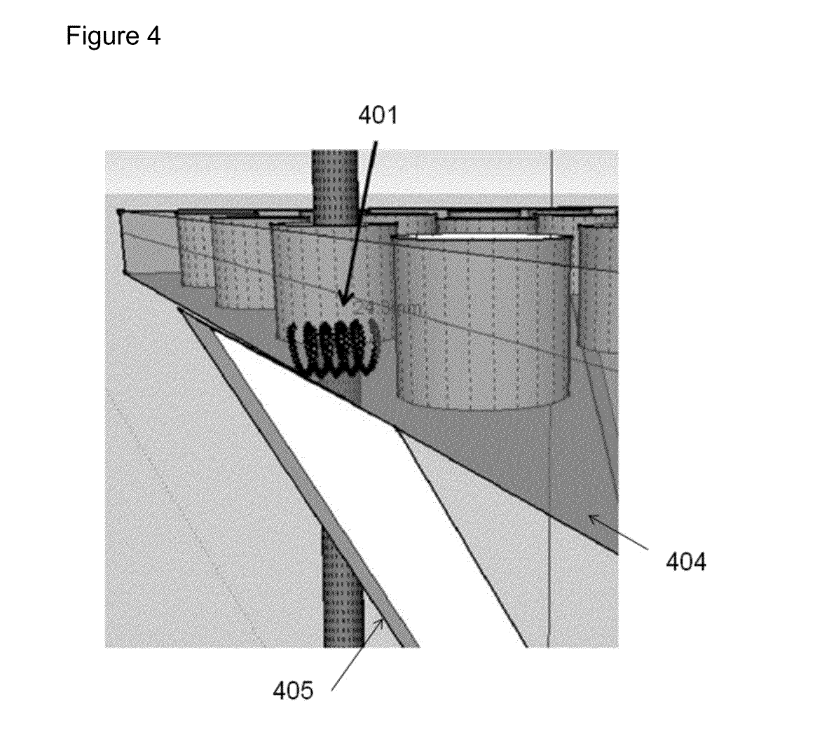

Some embodiments of the disclosure further comprise springs. Blocks and can be in contact with springs. Legs can be in contact with springs. FIG. 4 depicts a configuration comprising a spring. A spring 401 may be in substantial contact with one or more legs 405. A spring may be in contact with a block 404. In some embodiments, placing a solid tissue or a subject in the device, the tension from the spring may restrict movement of the solid tissue or subject. Restriction of movement may take place during needle insertion and/or agent injection.

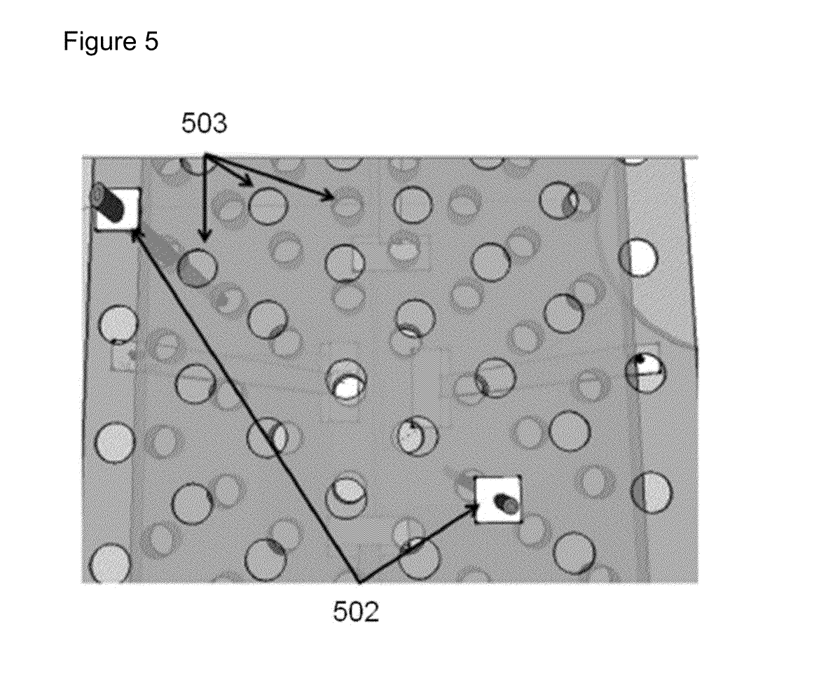

A control attachment can control movement of the blocks. FIG. 5 depicts a top view of a control attachment 502 in contact with a block 503. The control attachment 502 may be substantially square and may be attached around the needles. Upon contacting the upper surface of holes in the top block 503, the control attachment 502 may stop further insertion of the needles. The position of attachment of control attachment 502 to the needle may control depth of insertion. The function of a control attachment can be to restrict insertion of a needle. The control attachment can be adjustable.

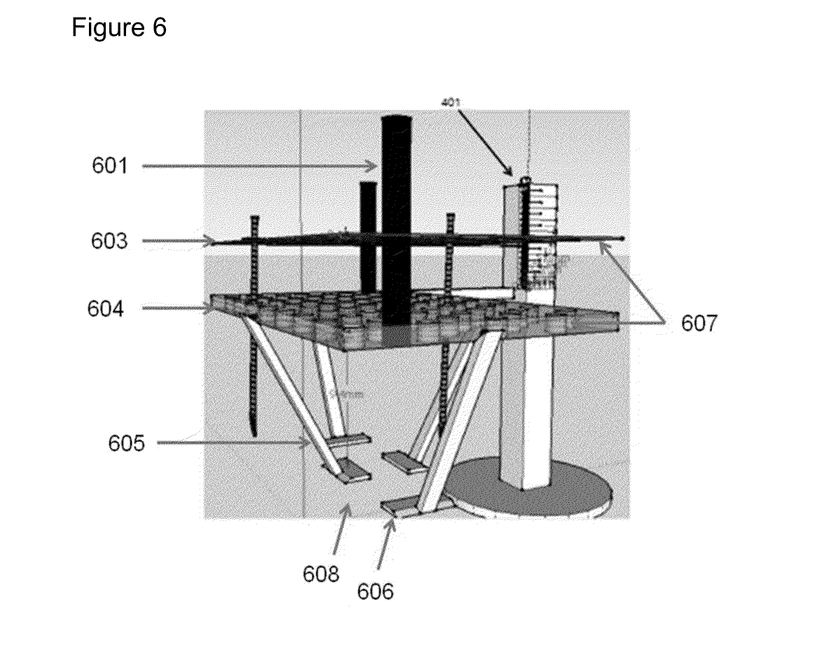

FIG. 6 depicts one particular type of device embodying principles of the present invention. In addition to the components outlined in FIG. 3, a system 601 for controlling vertical movement of the top block 603 is shown. Without being limited to a particular theory, the system 601 may serve to: (1) set the position of the top block 603 before needle insertion; (2) withdraw the top block 603 and needle away from a solid tissue or subject at a controllable speed.

The system 601 may set the position of the top block 603. A variety of factors, including the length of needle, the height of the bottom block 604, the size of a solid tissue and the depth of intended insertion, may be considered to determine a suitable position for the top block 603. The distance between the top block 603 and the bottom block 604 may not particularly limited. The distance may be 0, or at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 7.0, 8.0, 9.0, 10.0, 15, or 20 mm. The distance may be less than 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 7.0, 8.0, 9.0, 10.0, 15, or 20 mm. Alternatively, the distance may be about 0, 0.01, 0.02, 0.03. 0.04, 0.05, 0.06, 0.07, 0.08, 0.09, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 7.0, 8.0, 9.0, 10.0, 15, or 20 mm. The top block 603 may be in direct contact with the bottom block 604. In this embodiment, the distance between the two blocks can be zero. After setting the position of the top block, the system 601 may keep the top block stationary. A solid tissue or a subject may be placed underneath the bottom block 604. The solid tissue or subject may be placed substantially within the boundary set by all legs. One side of the solid tissue or subject may be placed against the bottom portion of leg 605 and/or the supporting floor 608. The other side may be placed against the bottom block 604 through adjusting legs 605. The placement of a solid tissue or a subject may occur before or after setting a suitable position for the top block 603. The platform may be adjusted to provide suitable stabilization for the solid tissue or subject. Needles can be inserted through holes in the top block 603 and the bottom block 604. The path of needle insertion may be guided by the holes. In some cases, the needles can be part of a needle array device.

A variety of types or the shapes of needle arrays can be employed. In some embodiments, the needle array can substantially or exactly match the configuration of holes defined by the top block and the bottom block. Furthermore, the present invention does not limit the type of needle to be used. A control attachment can be configured to be attached to the needle.

In some embodiments, needles can be a variety of sizes. In other embodiments, needles are of a particular size to function with the block holes. Needle size can be, for example, gauge 2, 4, 6, 8, 10, 12, 14, 16, 18, 20, 21, 22, 23, 24, 25, 26, 27, 28, 29 or 30, 31, 32, 33, or 34. Needles can be smaller than gauge 34. Needles can be standard commercially available needles. Needles can be end-port needles. The needles can be porous needles. The needles may be end-port and porous needles. One or more needles can be of the same gauge. One or more needles can be attached to a device. A plurality of needles can be positioned in an array.

The top block 603 can adjusted away from the bottom block 604. The adjustment can occur at a selected speed. The speed can be controlled by the system 601. In some embodiments, the bottom block can be adjusted from the top block.

One or more agents can be injected. In some embodiments, the same agent is injected through one or more needles. In some embodiments, different agents are injected through one or more needles. In some embodiments reference to one or more agents can mean the same agent at different concentrations.

Injection of agents can occur through needles. The agent can be injected into a location within a tissue. The injection of an agent can be simultaneous with the movement of the top or bottom block. The rate of lifting the block 603 and the rate of injection can be independently controlled. In some embodiments, the movement of a block is coupled to the movement of the needles. The choice of each rate can be determined by a variety of factors, such as for example, the type of solid tissue, the size of needle, the viscosity of the solvent and the permeability of the at least one agent. In some embodiments, the rate of movement of the block can beat least 0.1, 0.15, 0.2, 0.25, 0.3, 0.35, 0.4, 0.45, 0.5, 0.55, 0.6, 0.65, 0.7, 0.75, 0.8, 0.85, 0.9, 1.0, 1.1, 1.2, 1.4, 1.6, 1.8, 2.0, 2.2, 2.4, 2.6, 2.8, 3.0, 4.0, 5.0, 6.0, 7.0, 8.0, 9.0, 10, 11, 12, 13, 14, 15, 18, or 20 mm/min. In some embodiments, the rate of movement of the block is less than 0.1, 0.15, 0.2, 0.25, 0.3, 0.35, 0.4, 0.45, 0.5, 0.55, 0.6, 0.65, 0.7, 0.75, 0.8, 0.85, 0.9, 1.0, 1.1, 1.2, 1.4, 1.6, 1.8, 2.0, 2.2, 2.4, 2.6, 2.8, 3.0, 4.0, 5.0, 6.0, 7.0, 8.0, 9.0, 10, 11, 12, 13, 14, 15, 18, or 20 mm/min. In some embodiments, the rate of movement of the block is about 0.1, 0.15, 0.2, 0.25, 0.3, 0.35, 0.4, 0.45, 0.5, 0.55, 0.6, 0.65, 0.7, 0.75, 0.8, 0.85, 0.9, 1.0, 1.1, 1.2, 1.4, 1.6, 1.8, 2.0, 2.2, 2.4, 2.6, 2.8, 3.0, 4.0, 5.0, 6.0, 7.0, 8.0, 9.0, 10, 11, 12, 13, 14, 15, 18, or 20 mm/min.

In some embodiments, the rate of injecting the at least one agent is at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.5, 1.8, 2.0, 2.5, 3.0, 3.5, 4.0, 4.5, 5, 6, 7, 8, 9, 10, 15, 20, 25, 30, 40, 50 μl/min or even more. In some other cases, the rate of injecting the at least one agent is less than 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.5, 1.8, 2.0, 2.5, 3.0, 3.5, 4.0, 4.5, 5, 6, 7, 8, 9, 10, 15, 20, 25, 30, 40 or 50 μl/min. In some other cases, the rate of injecting the at least one agent is about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.5, 1.8, 2.0, 2.5, 3.0, 3.5, 4.0, 4.5, 5, 6, 7, 8, 9, 10, 15, 20, 25, 30, 40 or 50 μl/min.

A tissue treated with the device array described herein can be resected immediately following delivery of one or more agents. Alternatively, a region of tissue may be left in its native environment for a period of time before being resected. For example, a tissue may be left in its native environment for 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72, 76, 82, 88, 94, 100 or more hours following delivery is thought to be generally sufficient for a tumor to exhibit a detectable response. In other cases, the wait period may be minutes, hours, days, or weeks.

A tissue may be imaged using known methods to precisely locate the target region of tissue prior to insertion of the needles. The region may be imaged repeatedly before and after delivery of the plurality of agents to the region of tissue. The number of repeats may be 2, 3, 4, 5, 6, 7, 8, 9, 10, 11, 12, 13, 14, 15, 16, 17, 18, 19, 20 or even more.

According to some embodiments, solid tissue into which at least one agent has been delivered may be subsequently resected from the subject and evaluated. For example, in a case where the target tissue is a cancerous tumor, the plurality of agents injected therein can include some agents whose efficacy or effect on such tumors is under investigation. By injecting the various agents in vivo then waiting a selected period before removing the tumor, the effect of the agents on the tumor in situ can be investigated. This can preserve the tumor microenvironment and can distinguish this method from current ex vivo or in vitro therapeutics evaluation methods. Assuming that the needles used are configured to deliver a substantially equal amount of agents at any given location along their length, the agent delivered by each of the needles may be evenly distributed to the surrounding tissue along the delivery axis on which the respective needle was positioned during the delivery of the agent to a solid tissue. Over time, each agent can permeate outward from its delivery axis to a greater or lesser degree, depending on factors such as, for example, the density of the surrounding tissue, the viscosity and composition of the agent, the wettability of the tissue by the respective agent, etc. Typically, the portions of the tissue into which the agents spread can be approximately column-shaped regions coaxial with the respective delivery axes.

Needle Array Devices and Methods of Injection

The present disclosure relates to novel methods of injecting an agent into a solid tissue, comprising: (a) inserting at least one needle into the solid tissue; and (b) simultaneously injecting at least one agent into and withdrawing the needles from the solid tissue. The methods described herein can be referred to as an “extrusion method”. The term “standard injection method” used herein can generally refer to an injection method wherein in there is no substantial retraction of needles during injection of an agent.

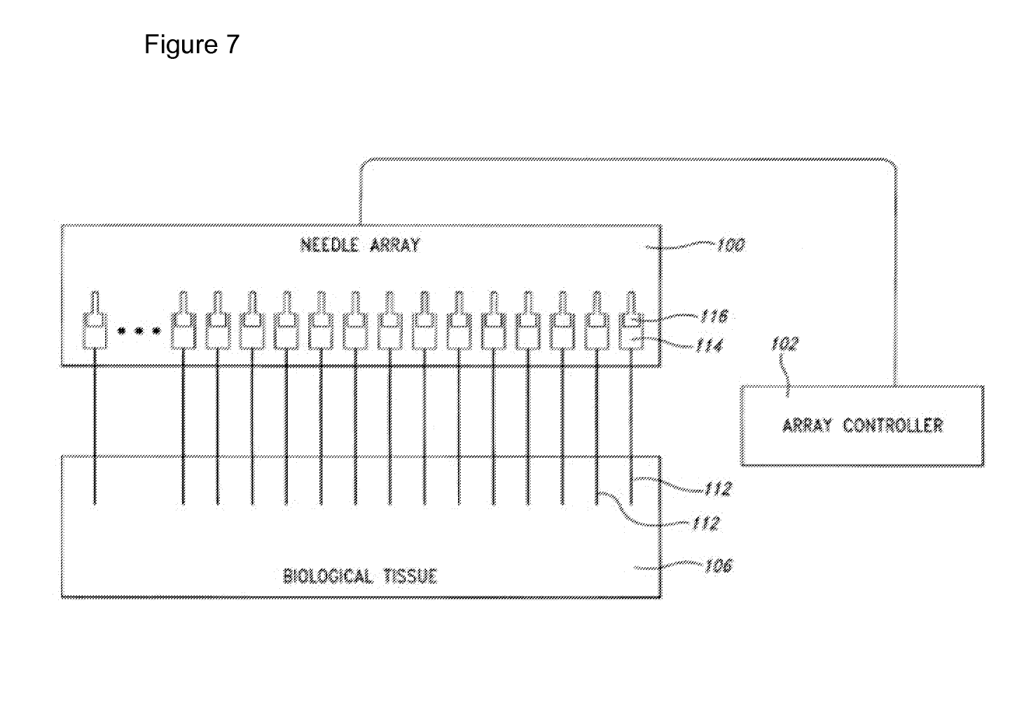

The needle array device can be used with two parallel blocks. The present invention may not limit the type or the shape of needle array so long as the shape of needle array matches the configuration of holes in the top block and the bottom block. Furthermore, the present invention may not limit the type of needle to be used as long as a control attachment is attached to the needle. Without being limiting, one example of a needle array device is shown in FIG. 7.

Referring to FIG. 7, a needle array assembly 700 is shown, including a plurality of needles 712, a plurality of reservoirs 714, a plurality of delivery actuators such as, in the present example, plungers 716, and a controller 702. Each of the plurality of needles 712 may be fixed in position relative to the others of the plurality of needles, and the plungers may be likewise operatively coupled so as to be fixed in position and simultaneously actuable. Each of the plurality of needles 712 may be in fluid communication with a respective one of the plurality of reservoirs 714, and each of the plurality of plungers can include a first end positioned in a respective one of the plurality of reservoirs 714. The controller 702 may be operatively coupled to second ends of each of the plurality of plungers 716. The controller 702 can be configured to control actuation of the plungers within the reservoir with respect to speed, distance, and direction of movement.

Needles may be inserted into a tissue simultaneously. Needles may be inserted into a tissue separately or individually. Without being bound by a particular theory or methods, needles may be inserted at slightly different times relative to one another to prevent distortion of the tissue. In some embodiments, when needles are inserted concomitantly or simultaneously, pressure from the needle insertion can cause compression of the tissue and result in distortion.

The present invention may not be limited by ways of loading agent(s) to reservoirs. In some cases as illustrated in FIG. 7, movement of the plurality of plungers 716 in a first direction may create a negative pressure in the respective reservoirs 714, drawing an agent or other fluid into the reservoirs via the respective needle 712, thereby charging the reservoirs. Each reservoir 714 can comprise a different agent, or some or all of the reservoirs can comprise a common agent. Movement of the plurality of plungers 716 in a second direction may create a positive pressure, or overpressure, in the respective reservoirs 714, forcing the contents of the reservoirs out via the respective needles 712. In this configuration, a relatively small amount of a plurality of agents can be simultaneously delivered directly to a region of solid tissue 706 for evaluation and analysis. In some embodiments, the amount of an agent delivered to the tissue may be less than 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, or 30 μl per needle. The evaluation of the tissue 706 and the efficacy of the different agents delivered thereto can be used, for example, to screen potential agents for subsequent clinical trials or to make patient-specific treatment decisions based on the relative efficacy of the agents in the tissue 706.

Agents may be loaded to the reservoir from the opposite end of the needle. Agents may be loaded by a pipette or the like. In some embodiments, the agents may be loaded under negative or positive pressure. In some embodiments, the agents may be loaded with a pump.

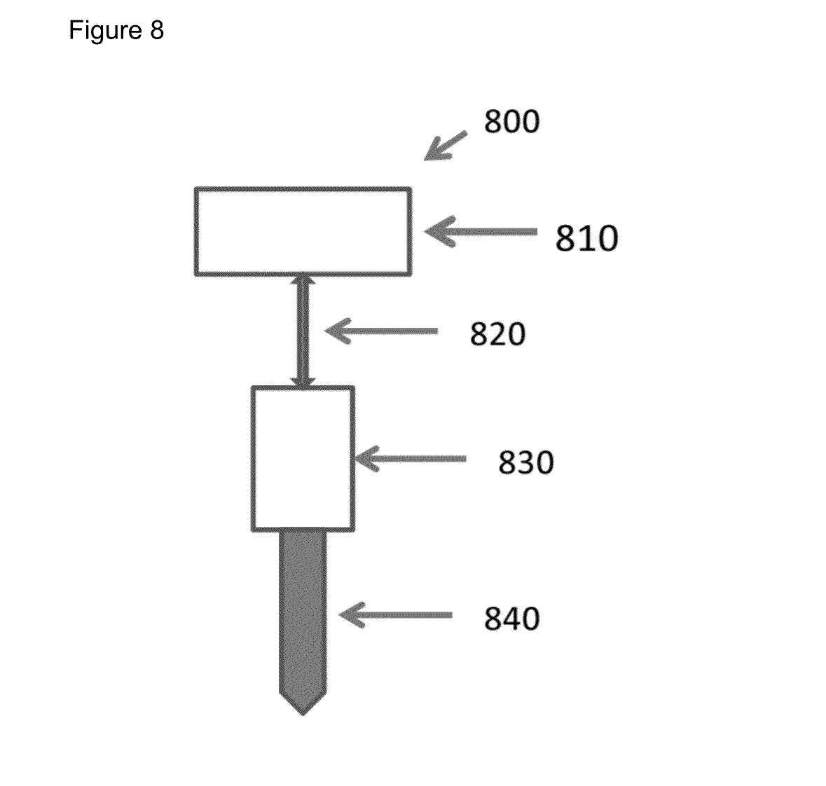

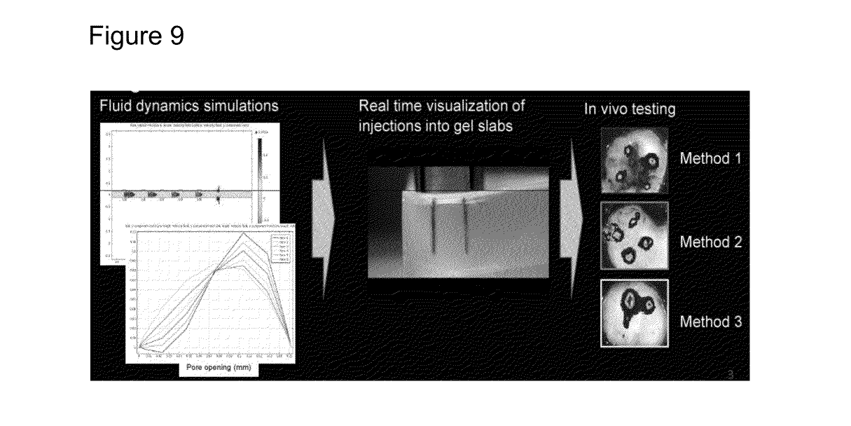

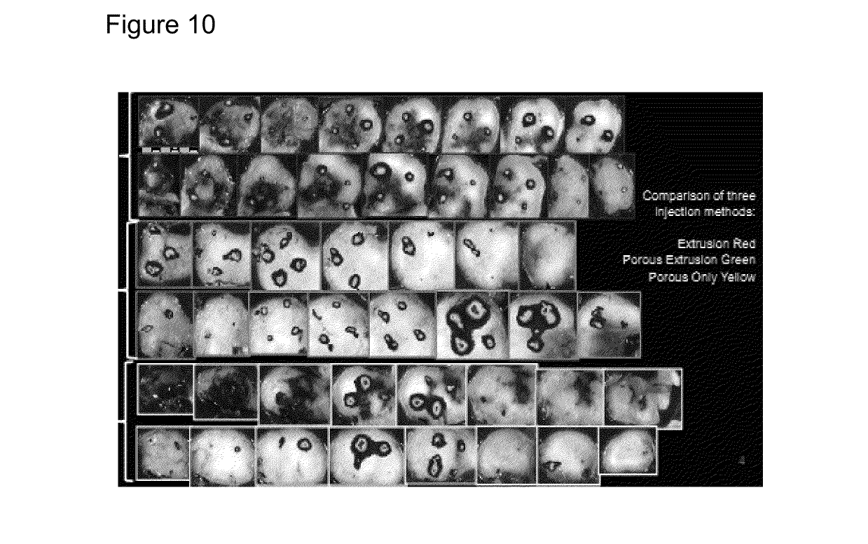

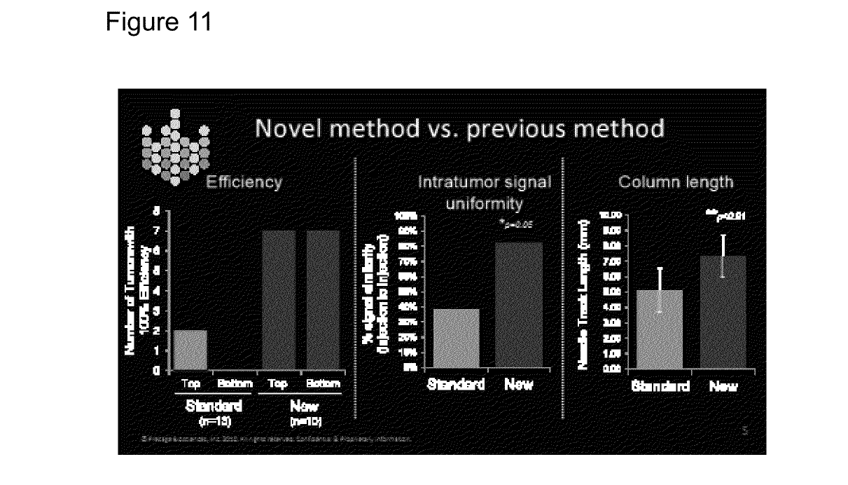

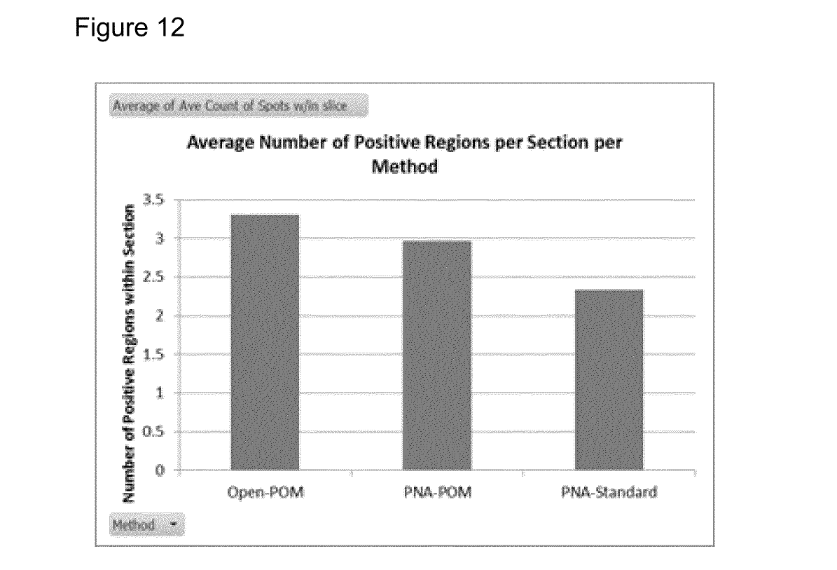

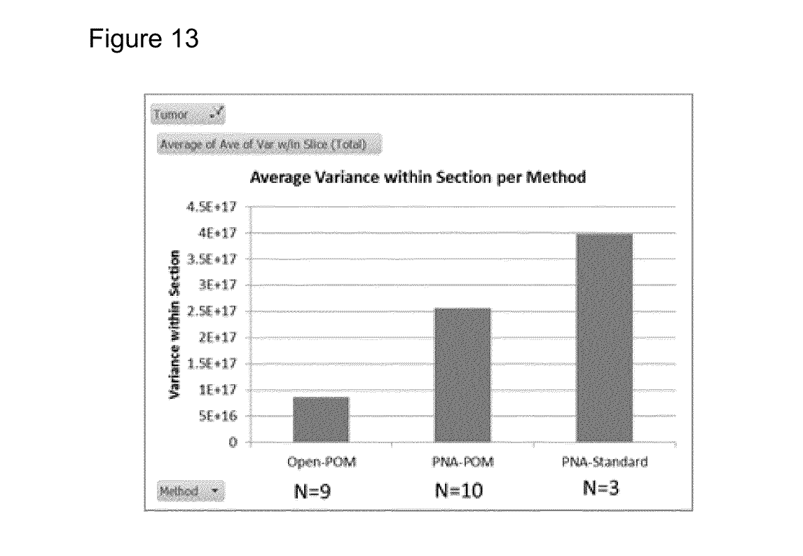

FIG. 8 shows an exemplary system and method of delivering agents to needles. The system 800 may be comprised of reservoir 810, solvent line 820, pump 830 and needle 840. The reservoir 810 may contain at least an agent admixed with at least one solvent and/or other excipients. The solvent line may be any tubular structure with a hollow structure inside for transporting the content in the reservoir. It may be made of metal or plastic. In some cases, the pump 830 may be a peristaltic pump. When the pump 830 is on, content in the reservoir can be transported from the reservoir 810 through the in-line pump 830 and ultimately delivered to the needle 840. The pump 830 may be directly connected to needle 840. Alternatively, there may be a solvent line connecting the pump 830 and the needle 840.

According to various embodiments, any number of needles can be used. For example, as few as one, two, three, four, five, six, seven, eight, nine or ten needles can be used, and according to some embodiments, more than one thousand needles can be used. In some embodiments, each of the needles may include a plurality of ports or apertures arranged along the length of the needle. The ports may be in pairs on opposite sides of the needle, the pairs being evenly spaced along its length. In addition, each pair may be rotated 90 degrees with respect to adjacent pairs of ports along the length of the needle. Alternatively, ports can be configured in such a way that they are largest near the tip-end of the needle, and the relative size of each of the plurality of ports can be inversely related to a distance of the respective port from the tip-end of the needle. Another possible configuration is that the ports closest to the tip-end of the needle are the most closely spaced, while the spacing between the ports grows increasingly greater as the distance from the tip-end increases. Yet another possible configuration of ports is that ports are formed in a spiral pattern, with each port rotated 90 degree with respect to adjacent ports.

In some embodiments, the device of the disclosure can comprise a placement needle. A placement needle can be in fluid communication with the reservoir of the device. The placement needle can determine the orientation of the device on the tumor. Determination of the correct orientation of the device on the tumor can occur before, during, or after injection of the one or more agents.

Some embodiments may contemplate direct drug delivery to a solid tissue at low flow rates with low shear forces that eliminate or reduce mechanochemical damage to tissues while permitting precisely targeted agent delivery to defined focal sites. These and related embodiments may permit selective delivery of an agent to a solid tissue in vivo in a therapeutically effective amount, while in some cases, the agent can be undetectable outside the solid tissue or is present at less than a minimal dose. Hence, problems (e.g., toxicity, detrimental side-effects, etc.) associated with administering excessively high systemic concentrations in order to obtain a therapeutically effective concentration in a desired solid tissue may be overcome.

Through the use of the methods described herein, which may include configuration (e.g., by placing at least one positional marker in one or more known locations) of the multiple needles in a manner that permits ready identification of the effects at a particular location, if any, of the contents released from a particular needle at the tissue location these and related embodiments thus contemplate methods of simultaneously delivering and comparing the relative therapeutic efficacies and/or toxicities of a large number of candidate agents. Such applications can find uses in drug screening and drug discovery, such as in preclinical animal models to identify and functionally characterize potential new therapeutics. For instance, a plurality of siRNAs can be administered and their relative abilities to knock down expression of a desired target gene can be compared. Other similar embodiments can find uses in clinical contexts, for example, to deselect, or eliminate from consideration, known agents that have no effect in a particular tumor, thereby advantageously advancing the therapeutic management of a subject by avoiding the loss of time and the undesirable side-effects that can be associated with administering an ineffectual treatment regimen.

Hand Held Devices

The present invention can be directed in certain embodiments as described herein to devices and methods for delivery of fluids to solid tissues, and in particular embodiments, to solid tumors with the use of a hand held device.

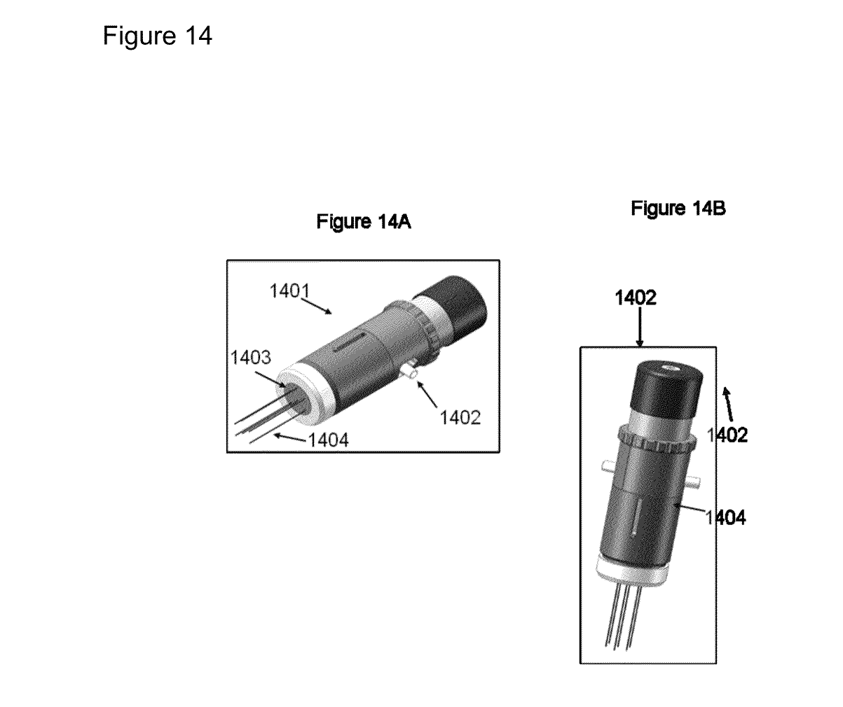

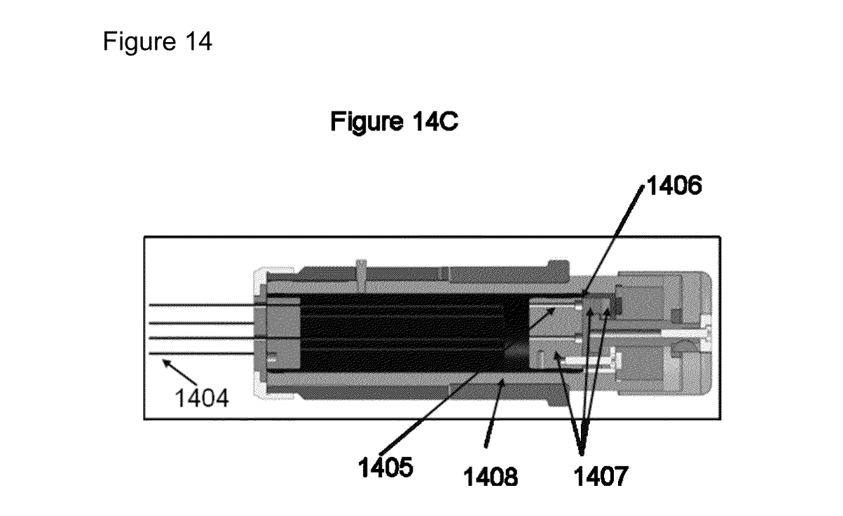

FIG. 14 depicts a delivery device 1401 for delivery of at least one agent to a solid tissue, comprising one or more knobs or one or more levers 1402 protruding from one or more sides of one end of the delivery device 1401 for controlling the movement of one or more needles 1404 and a block 1403 that can comprise a plurality of holes 1403 through which the needles can pass at an opposite end of the device. The one or more knobs or one or more levers 1402 can be coupled to the one or more needles 1404 within the delivery device 1401. The one or more needles 1404 can be coupled to a housing 1408 within the device. The one or more knobs or one or more levers 1402 can be coupled to the housing 1408 coupled to the one or more needles 1404. In some embodiments there can be two knobs or levers wherein each knob or lever can be coupled to the one or more needles 1404 such that movement of either or both knobs or levers 1402 controls movement of the one or more needles 1404. The one or more knobs or one or more levers 1402 can be coupled to the housing 1408 wherein the one or more needles are coupled such that movement of the one or more knobs 1402 results in movement of the housing 1408 and thus the one or more needles 1404. In some embodiments, the device can be handheld. In other embodiments, the device can be configured to rest on a solid surface or an apparatus that touches a solid surface such as a tripod.

The plurality of holes in the block 1403 may guide the insertion of a needle 1404. The size of the holes may be controlled to allow needles of a certain size to pass through. The holes may be arranged in an array wherein the holes may be arranged along substantially parallel axes. Each needle may have a plurality of ports, and the ports can be arranged to deliver a substantially equal amount of fluid at any given location along its length. Each needle can be an end-port needle having only one port. Each needle can be held in a bore 1405. The bore 1405 can support each needle 1404 and can provide a track for the movement of the needle. A needle held in the bore 1405 can be coupled to a plunger structure 1406 that can facilitate injection of the one or more needles 1404 into the tumor and can also facilitate withdrawal of the one or more needles 1404 out of the solid tissue.

The fluid delivery device can include one or more needles, each in fluid communication with a respective reservoir 1407. The one or more needles 1404 can each comprise one end which can connect to the respective reservoir 1407 and a second end through which one or more agents can be loaded. In another aspect, the second end of the one or more needles can be inserted into a solid tissue. In yet another aspect, a proximal end of the one or more needles can be in fluid communication with a respective reservoir 1407 and a distal end of the one or more needles can be used for loading one or more agents and/or can be inserted into a solid tissue. One or more respective plungers 1406 can be coupled to the respective reservoirs 1407 so as to be operable to drive fluid from the reservoirs via needle ports. A plunger 1406 can be coupled to each reservoir 1407 so as to be operable to drive fluid from each reservoir simultaneously. The needles 1404 can be advanced, one, two, three, four, five, six, seven, eight, nine, ten, or more at a time, in order to minimize the cumulative insertion force that would otherwise be required if multiple needles were inserted into the solid tissue simultaneously. The movement of the one or more plungers 1406 may be controlled by the one or more knobs or one or more levers 1402 protruding from the one or more sides of the fluid delivery device.

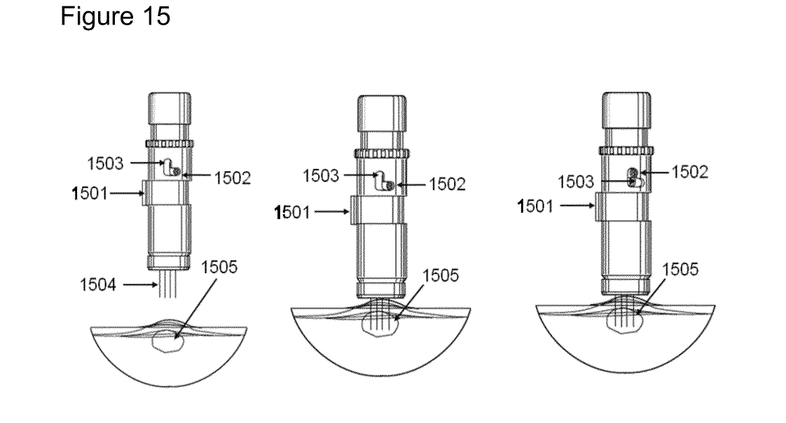

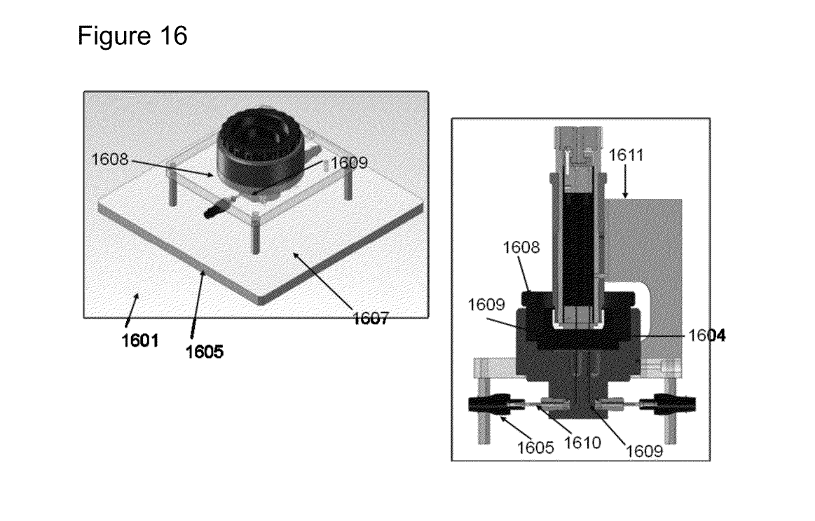

FIG. 15 depicts an exemplary method of agent delivery to a tumor. The device can be loaded with one or more agents. The device can be inserted into a solid tissue 1505. Injection of the one or more agents can occur by retracting the needles 1504, a process which can deposit the agents into the tissue 1505. Each step can be controlled by one or more knobs or one or more levers 1502. The one or more knobs or one or more levers 1502 can be housed in a track 1503 that can allow the one or more knobs or one or more levers 1502 to be moved between multiple positions along the length of the track 1503. The one or more knobs or one or more levers 1502 can be moved into a position along the track that can lock the one or more knobs or one or more levers 1502 in a specific position 1503. The one or more knobs or one or more levers 1502 can be moved into a position along the track 1503 that can allow the one or more knobs or one or more levers 1502 to move freely within the track 1503. The one or more knobs or one or more levers 1502 can be moved into the locked position in the track 1503 while one or more agents are loaded into the device and moved into the unlocked position of the track 1503 for delivery of the one or more agents into a target tissue. The one or more knobs or one or more levers 1502 can be in the locked position during loading of the one or more agents into the device and during the insertion of the one or more needles 1504 into the target tissue 1505, and then moved to the unlocked position once the one or more needles 1504 have been inserted into the target tissue 1505. When the one or more knobs or one or more levers 1502 is in the locked position, the one or more needles can be fully extended. When the one or more knobs or one or more levers 1502 is in the unlocked position, the one or more knobs or one or more levers 1502 can be further moved within the track 1503 which can cause the one or more needles 1504 to retract. Following the insertion of the one or more needles 1504 of the device into the target tissue, the one or more knobs or one or more levers 1502 can be moved into the unlocked position and further moved within the track 1503 which can cause the one or more needles 1504 to retract. In one aspect, the retraction of the one or more needles 1504 can cause the delivery of one or more agents from the one or more needles simultaneously along respective axes or columns within the tissue during retraction of the one or more needles 1504. The movement of the one or more knobs or one or more levers 1502 can be used to selectively control the rate and volume of fluid through the needles 1504.

The fluid delivery device of the present invention can further comprise a controller. The controller can be in communication with the one or more reservoirs connected to the one or more needles. The controller can be in communication with the one or more plungers. The controller can be coupled to the one or more plungers. The controller can control the movement of the one or more needles. The controller can be the one or more knobs or one or more levers coupled to the one or more plungers. The controller can be coupled to the one or more knobs or one or more levers coupled to the one or more plungers. The controller can control movement of the one or more knobs or one or more levers coupled to the one or more plungers. The controller can control the rate of withdrawal of the one or more needles. The controller can control the rate of delivery of one or more agents. The controller can control the rate of delivery of one or more agents by controlling the movement of the one or more needles. The controller can control the rate of delivery of one or more agents by controlling the withdrawal of the one or more needles. The controller can comprise a motor. The controller can comprise an individual operating the device of the present invention. The controller can comprise an electronic device. The electronic device can be a computer. The controller can be a stepper motor. The stepper motor can be in communication with a lead screw.

The fluid delivery device of the present invention can optionally further comprise one or more additional chambers that can be in communication with the one or more reservoirs connected to the one or more needles and can serve as a venting mechanism. The venting mechanism can be a deairification mechanism. The one or more additional chambers can facilitate the release of bubbles from the one or more reservoirs connected to the one or more needles while the reservoirs are being loaded with one or more agents. The bubbles can be air bubbles. The venting mechanism described herein can be used such that each of the one or more reservoirs connected to the one or more needles can contain substantially reduced amounts of bubbles. The venting mechanism described herein can be used such that each of the one or more reservoirs connected to the one or more needles can contain no bubbles. The venting mechanism described herein is a closed system. The closed system equalizes the pressure in the transfer assembly.

The fluid delivery device of the present invention can further comprise one or more depth control mechanisms 1501. The depth control mechanism can comprise an elongated piece that can be coupled with the end of the device that comprises a plurality of holes through which the one or more needles pass. The elongated piece can comprises a plurality of holes that can directly couple with the plurality of holes on the end of the delivery device such that the one or more needles 1504 can pass through the plurality of holes on the end of the device as well as through the plurality of holes in the elongated piece. The elongated piece can act to extend the length of the device. In this aspect, the elongated piece can be used to limit the length of the one more needles that can be inserted into a solid tissue.

The device can simultaneously deliver a plurality of fluid agents along respective axes in solid tissue in vivo. The device can comprise a stroke volume that can control the size of the delivery column of the plurality of fluid agents. The stroke volume can allow a delivery column of an agent between 0.1 and 100 millimeters (mm). The stroke volume can allow a delivery column of an agent that is at least about 0.1, 0.5, 1, 2, 3, 4, 5, 6, 7, 8, 9, 10, 20, 30, 40, 50, 60, 70, 80, 90 100 or millimeters. The stroke volume can allow a delivery column of an agent that is at most about 0.1, 0.5, 1, 2, 3, 4, 5, 6, 7, 8, 9, 10, 20, 30, 40, 50, 60, 70, 80, 90 100 or millimeters. In some instances, the stroke volume can allow a delivery column of an agent between about 1-10 mm.

The elongated piece can be in contact with the solid tissue 1505 or the skin over the solid tissue. In another embodiment, the depth control mechanism 1501 can comprise a clip that can be inserted along the length of the body of the delivery device between the one or more knobs or one or more levers protruding from the side of the device and the end of the device that comprises the plurality of holes through which the one or more needles pass. In this aspect, the clip serves to lengthen the body of the device such that the length of the one or more needles protruding from the end of the end comprising the plurality of holes is reduced. In yet another embodiment, the depth control mechanism comprises the elongated piece and clip described herein. In this aspect, the length of the one or more needles protruding from the end of the device can be reduced further than the use of either the elongated piece or the clip described herein used alone.

In yet another aspect, the fluid delivery device of the present invention comprises a depth control 1501 mechanism wherein the length of the one or more needles 1504 that can be inserted into a solid tissue can be controlled individually. In this aspect, the depth control mechanism can be used to alter the length of 1 needle, 2 needles, 3 needles, 4 needles, 5 needles, 6 needles, 7 needles, 8 needles, 9 needles, 10 needles, 50 needles, 100 needles, 500 needles, or 1000 needles. The depth control mechanism can be used to alter the length of any 1 needle, any 2 needles, any 3 needles, any 4 needles, any 5 needles, any 6 needles, any 7 needles, any 8 needles, any 9 needles, any 10 needles, any 50 needles, any 100 needles, any 500 needles, or any 1000 needles. In some instances the needle depth can be altered such that one or more needles are inserted into different depths.

The present invention does not limit the type of needle to be used. The needle can pass through one of the plurality of holes in the end of the delivery device that comprises the plurality of holes and can be securely coupled to the one or more reservoirs within the delivery device. The size of the plurality of holes can be independently controlled to allow needles of specific gauge to pass through them. When the size of the holes is uniform, needles of the same size are used. When the size of the holes is different, needles of different sizes are used. In a further aspect, the holes in the end of the device comprising a plurality of holes may have a variety of arrangements. The holes can form substantially parallel rows. The number of holes can be controlled to allow a specific number of needles to be inserted. The number of hole(s) can be 1, 2, 3, 4, 5, 6, 7, 8, 9, 10 or more. The number of holes can equal the number of needles. The number of holes may be greater than 10. The number of holes may be greater than 100. The number of holes may be greater than 500. The number of holes can be 6. The number of holes can be 10. The number of reservoirs within the delivery device can be equal to the number of holes. The number of reservoir(s) could be 1, 2, 3, 4, 5, 6, 7, 8, 9, 10, or more. The number of reservoirs can equal the number of needles and may be greater than 10. The number of reservoirs may be greater than 100. The number of reservoirs may be greater than 500. The number of reservoirs can be 6. The number of reservoirs can be 10. Any of the needles may be independently selected from gauge 2, 3, 4, 5, 6, 7, 8, 9, 10, 11, 12, 13, 14, 16, 18, 20, 21, 22, 23, 24, 25, 26, 27, 28, 29, 30, 32, 34, 36, 38, 40, 42, 44, 46, 48 49, 50 or more. The needles may be end-port needles or porous needles. The needles can be end-port needles. The needles can be porous needles. The needles can be a mixture of end-port needles and porous needles. In some cases, all the needles are gauge 25. In some embodiments, all of the needles are end-port needles.

The loading of one or more agents into the one or more needles of the fluid delivery device of the present invention can be achieved in any manner known in the art for loading an agent into a syringe comprising a needle. In one aspect, one or more agents can be loaded into the second end or distal end of the one or more needles. The one or more needles of the fluid delivery device can be loaded with the same agent. The one or more needles of the fluid delivery device can be loaded with different concentrations of the same agent. The one or more needles of the fluid delivery device can be loaded with a different agent. Different agents can be loaded into two or more, three or more, four or more, five or more, six or more, seven or more, eight or more, nine or more, ten or more, 100 or more, 500 or more, or 1000 or more needles of the fluid delivery device. The same agent can be loaded into two or more, three or more, four or more, five or more, six or more, seven or more, eight or more, nine or more, ten or more, 100 or more, 500 or more, or 1000 or more needles of the fluid delivery device. In one embodiment combinations of different agents are loaded into different needles. In one embodiment, different concentrations of the same agent can be loaded into two or more, three or more, four or more, five or more, six or more, seven or more, eight or more, nine or more, ten or more, 100 or more, 500 or more, or 1000 or more needles of the fluid delivery device.

The loading of one or more agents into the one or more needles of the fluid delivery device of the present invention can be achieved through the use of a loading mechanism. The loading mechanism can comprise a transfer assembly 301. The transfer assembly can be used to load the one or more needles of the fluid delivery device with the same agent. The transfer assembly can be used to load the one or more needles of the fluid delivery device with different concentrations of the same agent. The transfer assembly can be used to load the one or more needles of the fluid delivery device with a different agent. The transfer assembly can be used to load different agents into two or more, three or more, four or more, five or more, six or more, seven or more, eight or more, nine or more, ten or more, 100 or more, 500 or more, or 1000 or more needles of the fluid delivery device. The transfer assembly can be used to load the same agent into two or more, three or more, four or more, five or more, six or more, seven or more, eight or more, nine or more, ten or more, 100 or more, 500 or more, or 1000 or more needles of the fluid delivery device. In one embodiment, the transfer assembly can be used to load different concentrations of the same agent into two or more, three or more, four or more, five or more, six or more, seven or more, eight or more, nine or more, ten or more, 100 or more, 500 or more, or 1000 or more needles of the fluid delivery device.

The transfer assembly 1601 can comprise a raised platform 1607. The raised platform can comprise a central reservoir 1608. The central reservoir 1608 can stabilize the body of the device during loading of agents into the needles 1604. The central reservoir 1608 can further comprise one or more needle reservoirs 1609 for housing the one or more needles 1604. Each needle reservoir 1609 can surround the one or more needles 1604 of the device. The transfer assembly 1601 can comprise one central reservoir 1608 and a needle reservoir 1609 for each needle 1604 present in the fluid delivery device of the present invention such that each needle reservoir 1609 can house one needle 1604. In one aspect, each needle reservoir of the transfer assembly can be loaded simultaneously with an agent. Each needle reservoir of the transfer assembly can be loaded individually with an agent. Each needle reservoir can be used to facilitate the delivery of an agent into the needle housed within the needle reservoir.

Each needle reservoir can comprise a first end for receiving a needle and a second end that can be in fluid communication with a channel 1610. Each channel can be in fluid communication with the end of each needle reservoir 1609. Each channel can be in fluid communication with the central reservoir 1608. The channel 1610 in fluid communication with the second end of the needle reservoir 1609 can be used to deliver an agent to the needle reservoir. The channel can comprise tubing. Each of the one or more needles of the fluid delivery device can be inserted into a needle reservoir such that the second end or distal end of the one or more needles can be housed at the second end of the needle reservoir and thus can be in fluid communication with the channel that can be used to deliver an agent to the needle reservoir. In another aspect of this embodiment, the needle housed in the needle reservoir can be loaded with the agent delivered to the needle reservoir through the channel in fluid communication with the second end of the needle reservoir.

Each channel can be coupled to an adaptor 1605. The adaptor can connect the channel 1610 and needle reservoir 1609 such that they can remain in fluid communication with each other. The adaptor 1605 can be a luer adaptor. The adaptor 1605 can comprise a closed system transfer device (CSTD) wherein a male luer adaptor can couple a driver and the channel in fluid communication with the needle reservoir to create a closed system. In this aspect, the CSTD creates a closed channel between the driver and the channel in fluid communication with the needle reservoir such that no agent can be released by the driver until the CSTD is connected to the channel in fluid communication with the needle reservoir. Agents can be loaded into the one or more needles of the device through the luer lock.

The luer lock can be connected to a driver that can deliver an agent through the channel to the needle reservoir. The driver can create pressure that can be used to drive an agent through the channel in fluid communication with the needle reservoir into the needle housed within the needle reservoir. In another aspect, each needle reservoir can be connected to a different driver. The driver can be in fluid communication with the channel in fluid communication with the needle reservoir. The driver can be a syringe. The driver can comprise a reservoir space, such as the central cylinder of a syringe that can comprise an agent. A reservoir of a driver can be filled with an agent and can be used to drive the agent through a channel in fluid communication with a needle reservoir that can be part of a transfer assembly such that the agent can be further driven from the needle reservoir into a needle of the fluid delivery device of the present invention. The pressure created by the driver can be used to drive bubbles out of the one or more needles of the fluid delivery device during loading of an agent into the one or more needles with the transfer assembly described herein.

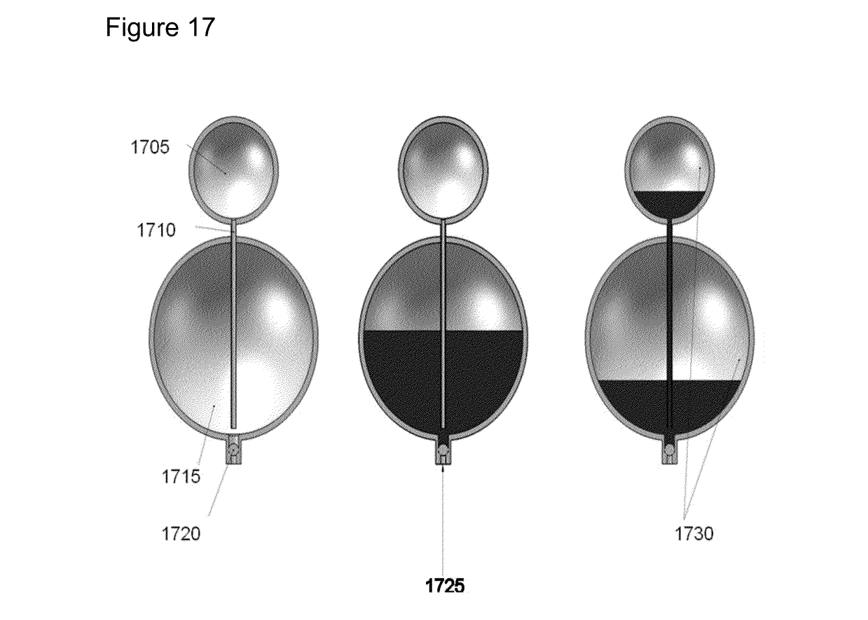

The pressure created by the driver can facilitate the insertion of the agent into the needle. FIG. 17 depicts an exemplary needle reservoir and agent loading mechanism of the disclosure. A needle reservoir can comprise two chambers, and upper chamber 1705 and a lower chamber 1715. The chambers can be connected by an interchamber passage 1710. The lower chamber can comprise a check valve 1720, which can serve as an agent loading point. Agents can be loaded through the check valve into the lower chamber using a driver. The pressure of injection of the agents by the driver can be enough to force the movement of agent into the needle, up through the interchamber passage and into the upper chamber 1730.

In some embodiments, the device can be depressed to create a vacuum. The vacuum can have a negative pressure. The negative pressure of the vacuum can facilitate loading of the agents into the needle reservoirs. In some instances, the vacuum is a partial vacuum. The agent can be loaded through the check valve 1720. Without being bound by theory, in some instances the air displaced by the agent in the lower chamber would be compressed, which could result in a higher pressure in the lower chamber. The higher pressure would result in an expansion of the air, which could force the agent into the upper chamber, thereby loading the agent into the needle.

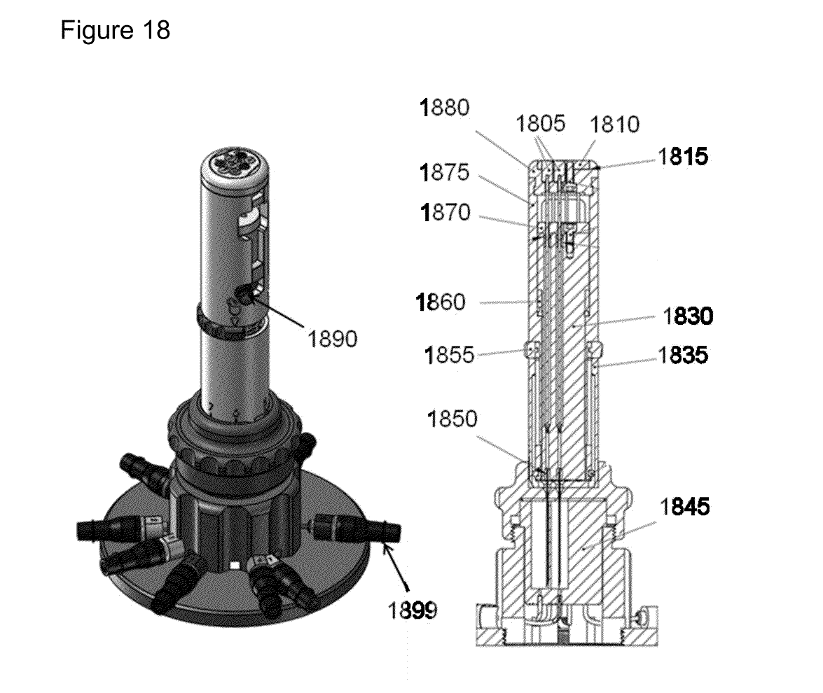

FIG. 18 shows an exemplary embodiment of the handheld device 1875 of the disclosure. The device comprises plungers 1805, a plunger cap 1810, and washer 1815. The device comprises a syringe body 1830 on top of a transfer vessel 1845. The device can also comprise a needle assembly 1850. The device can also comprise a needle guard 1835. The device can also comprise a needle stop collar 1855. The device can also comprise a slide lock ring 1860. The device can also comprise a rod seal retainer 1870. The device can comprise a device cap 1880. The device can also comprise locks 1890. The device can also comprise adaptors 1899 that facilitate loading of agents into the device.

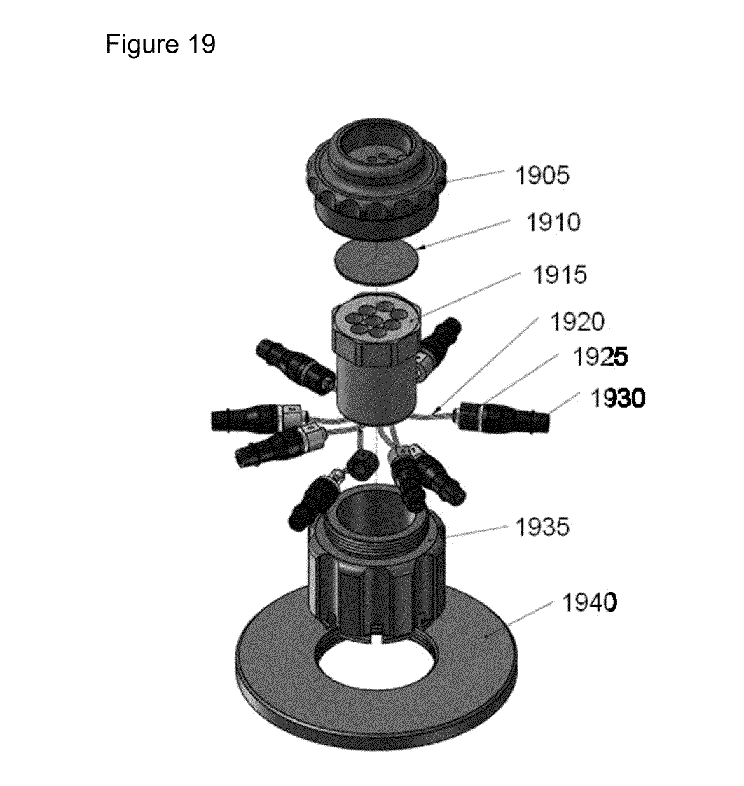

FIG. 19 depicts another exemplary embodiment of the device of the disclosure. The device can comprise a transfer vessel needle guide 1905. The device can comprise a septa 1910 on top of a transfer vessel body 1915. Connected to the transfer vessel body are channels 1920. Channels can be connected to an adaptor 1930. An adaptor can comprise a microclave ID collar 1925. The device can be stabilized by a threaded retainer 1925 that can be housed in a transfer vessel base 1940.

According to some embodiments, a solid tissue into which at least one agent has been delivered is subsequently resected from the subject and evaluated. For example, in a case where the target tissue is a cancerous tumor, the plurality of agents injected therein can include some agents whose efficacy or effect on such tumors is under investigation. The plurality of agents can include microdoses of non-FDA approved agents or investigational agents. By injecting the various agents in vivo then waiting a selected period before removing the tumor, the effect of the agents on the tumor in situ can be investigated. This preserves the tumor microenvironment and distinguishes this method from current ex vivo or in vitro therapeutics evaluation methods. Assuming that the needles used are configured to deliver a substantially equal amount of agents at any given location along their length, the agent delivered by each of the needles is evenly distributed to the surrounding tissue along the delivery axis on which the respective needle was positioned during the delivery of the agent to a solid tissue. Over time, each agent permeates outward from its delivery axis to a greater or lesser degree, depending on factors such as, for example, the density of the surrounding tissue, the viscosity, polarity, hydrophobicity, and composition of the agent, the wettability of the tissue by the respective agent, etc. Typically, the portions of the tissue into which the agents spread are approximately column-shaped regions coaxial with the respective delivery axes.

According to various embodiments, a region of tissue is left in place for some period of time before being resected. For example, a tumor may be resected, between 1 and 72 hours following delivery. In other cases, the wait period may be minutes, hours, days, or weeks. In some instances, 24 hours following delivery sufficient for a tumor to exhibit a detectable response. In addition, the tissue region may be imaged using known methods to precisely locate the target region of tissue prior to insertion of the needles. The region may be imaged repeatedly before and after delivery of the plurality of agents to the region of tissue. The number of repeats may be 2, 3, 4, 5, 6, 7, 8, 9, 10, 11, 12, 13, 14, 15, 16, 17, 18, 19, 20 or even more.

The fluid delivery device described herein can be used for precise control of the depth of needle insertion and also provide exquisite control of the delivery of at least one agent to a solid tissue.

In a preferred embodiment, the device leads to precise microinjection of multiple agents directly into solid tumors. If thereafter resected, the tissue can be sectioned for evaluation of an effect of each agent on the tissue, and based on the evaluation, candidate agents selected, deselected, or prioritized for clinical trials or therapy, and subjects selected, deselected, or prioritized for clinical trials or therapeutic treatment.

Agents

In certain other embodiments, the agents comprise an agent that is selected from (a) a gene therapy agent; (b) a chemotherapy agent; (c) a small molecule; (d) an antibody; (e) a protein; (f) one of a small interfering RNA and an encoding polynucleotide therefor; (g) one of an antisense RNA and an encoding polynucleotide therefor, (h) one of a ribozyme and an encoding polynucleotide therefor; (i) a detectable label; (j) one of a therapeutic protein, polypeptide, and a peptidomimetic; (k) and antibody-drug conjugates. In certain further embodiments, the detectable label is selected from a radiolabel, a radio-opaque label, a fluorescent label, a colorimetric label, a dye, an enzymatic label, a GCMS tag, avidin, and biotin. In certain embodiments, the agents are selected from (i) a gene therapy agent that comprises at least one operably linked promoter, (ii) a small interfering RNA-encoding polynucleotide that comprises at least one operably linked promoter; (iii) an antisense RNA encoding polynucleotide that comprises at least one operably linked promoter; and (iv) a ribozyme-encoding polynucleotide that comprises at least one operably linked promoter. In certain further embodiments, the operably linked promoter is selected from a constitutive promoter and a regulatable promoter. In certain still further embodiments, the regulatable promoter is selected from an inducible promoter, a tightly regulated promoter and a tissue-specific promoter. In certain still further embodiments, the regulatable promoter is selected from an inducible promoter, a tightly regulated promoter and a tissue-specific promoter. Example of anti-angiogenic agent includes, but is not limited to, bevacizumab and others in development. Example of epigenetic modifier includes, but is not limited to, azacitididne and decitabine and others in development. The agent may be a small molecule agent with significant cytotoxicity. The agent may include ubiquitin activating enzyme inhibitors and proteasome inhibitors such as, bortezomib and ixazomib citrate.

Agents may be dissolved or suspended in an aqueous solution as a mixture or colloid that may be delivered to a solid tissue. When used to refer to agent delivered through a needle, the term agent is to be read broadly on any substance capable of being delivered through a needle, including liquids, gases, colloids, suspended solids, etc.

In some embodiments, the agents are marketed anti-cancer drugs. Marketed anti-cancer drugs include, but are limited to, Lomustine, Carmustine, Streptozocin, Mechlorethamine, Melphalan, Uracil Nitrogen Mustard, Chlorambucil, Cyclophosphamide, Iphosphamide, Cisplatin, Carboplatin, Mitomycin, Thiotepa, Dacarbazin, Procarbazine, Hexamethyl Melamine, Triethylene Melamine, Busulfan, Pipobroman, Mitotane, Methotrexate, Trimetrexate, Pentostatin, Cytarabine, Ara-CMP, Fludarabine phosphate, Hydroxyurea, Fluorouracil, Floxuridine, Chlorodeoxyadenosine, Gemcitabine, Thioguanine, 6-Mercaptopurine, Bleomycin, Topotecan, Irinotecan, Camptothecin sodium salt, Daunorubicin, Doxorubicin, Idarubicin, Mitoxantrone, Teniposide, Etoposide, Dactinomycin, Mithramycin, Vinblastine, Vincristine, Navelbine, Paclitaxel, Docetaxel. In some embodiments, the agents are candidate oncology agents. Candidate oncology agents can be selected from resources that disclose listings of investigational therapeutics, for instance, the National Institutes of Health (Bethesda, Md.) which maintains a database of ongoing and planned clinical trials at its “ClinicalTrials.gov” website.

Agents refer to any fluid or molecule in an aqueous solution, mixture, or colloid that may be delivered to a target tissue. When used to refer to agent delivered through needles, the term agent is to be read broadly to read on any substance capable of flowing through such a microdialysis probe or needle, including liquids, gases, colloids, suspended solids, etc.

In some embodiments, the agents are active forms of a prodrug. Prodrugs can be converted to their active form normally by natural metabolic processes. Prodrugs can be classified as Type I or Type II. Type I prodrugs are activated intracellulary. Type I prodrugs can include nucleoside analogs, idoxurine, 5-fluorouracil, 5-fluorocytosine, ganciclovir, Acyclovir, trifluorothymidine, adenine arabinoside bromovinyldeoxyuridine, penciclovir, diethylstilbestrol diphosphate, cyclophosphamide, L-dopa, 6-mercaptopurine, mitomycin C, zidovudine, Carbamazepine, captopril, carisoprodol, heroin, molsidomine, paliperidone, phenacetin, primidone, psilocybin, sulindac, and fursultiamine, MTX-α-peptide. Type II prodrugs are activated extracellularly. Type II prodrugs can include Lisdexamfetamine, loperamide oxide, oxyphenisatin, sulfasalazine, Acetylsalicylate, bacampicillin, bambuterol, chloramphenicol succinate, dihydropyridine pralidoxime, dipivefrin, and fosphenyloin. Chemotherapeutic prodrug therapy can include antibody-directed enzyme prodrug therapy (ADEPT), virus-directed enzyme prodrug therapy (VDEPT), gene-directed enzyme prodrug therapy (GDEPT), Clostridial-directed enzyme prodrug therapy (CDEPT). Prodrugs can be linked to nanoparticles or lipsosomes.

Agents for use in screening methods and in methods of rating candidate agents for development into therapeutic agents can be provided as “libraries” or collections of compounds, compositions or molecules. Such molecules typically include compounds known in the art as “small molecules” and having molecular weights less than 10⁵ daltons, less than 10⁴ daltons, or less than 10³ daltons.

For example, a plurality of members of a library of test compounds can be introduced as therapeutic agents to a region of a solid tumor of known tumor type in each one or a plurality of subjects having a tumor of the known tumor type, by distributing each of the therapeutic agents to a plurality of positions along an axis within the region in each subject, and after a selected period of time (e.g., a range of time, a minimum time period or a specific time period) the region of solid tumor in which the candidate agents have been introduced can be imaged or removed from each subject, and each region compared by detecting an effect (if any) of each agent on the respective position within the region, for instance, by determining whether an altered physiologic state is present as provided herein, relative to positions in the region that are treated with control agents as provided herein, which would either produce no effect (negative control) or a readily detectable effect (positive control).

Agents further can be provided as members of a combinatorial library, which can include synthetic agents prepared according to a plurality of predetermined chemical reactions performed in a plurality of reaction vessels. For example, various starting compounds can be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides or other compositions that can include small molecules. Those having ordinary skill in the art will appreciate that a diverse assortment of such libraries can be prepared according to established procedures, and tested for their influence on an indicator of altered mitochondrial function, according to the present disclosure. Other agents can be proteins (including therapeutic proteins), peptides, peptidomimetics, polypeptides, and gene therapy agents (e.g., plasmids, viral vectors, artificial chromosomes and the like containing therapeutic genes or polynucleotides encoding therapeutic products, including coding sequences for small interfering RNA (siRNA), ribozymes and antisense RNA) which in certain further embodiments can comprise an operably linked promoter such as a constitutive promoter or a regulatable promoter, such as an inducible promoter (e.g., IPTG-inducible), a tightly regulated promoter (e.g., a promoter that permits little or no detectable transcription in the absence of its cognate inducer or derepressor) or a tissue-specific promoter. Methodologies for preparing, testing and using these and related agents are known in the art.

In some embodiments, the agent is a small molecule agent. As used herein, the term “small molecule agent” means an agent with a molecule weight less than about 1000 daltons, less than about 800 daltons, or less than about 500 daltons. In some further embodiments, the small molecule agent is an anti-cancer agent. The anti-cancer agent may be an approved anti-cancer drug currently on the market, an anti-cancer drug currently in clinical trials, an anti-cancer drug withdrawn from clinical trials or market due to toxicity or lack of efficacy, or an early stage anti-cancer drug in the development.

Pharmaceutically acceptable carriers for therapeutic use are well known in the pharmaceutical art. For example, sterile saline and phosphate-buffered saline at physiological pH can be used. Preservatives, stabilizers, dyes and other ancillary agents can be provided in the pharmaceutical composition. For example, sodium benzoate, sorbic acid and esters of p-hydroxybenzoic acid can be added as preservatives. In addition, antioxidants and suspending agents can be used. “Pharmaceutically acceptable salt” refers to salts of drug compounds derived from the combination of such compounds and an organic or inorganic acid (acid addition salts) or an organic or inorganic base (base addition salts). The agents, including drugs, contemplated for use herein can be used in either the free base or salt forms, with both forms being considered as being within the scope of the certain present invention embodiments.

Other agents can be antibodies, including naturally occurring, immunologically elicited, chimeric, humanized, recombinant, and other engineered antigen-specific immunoglobulins and artificially generated antigen-binding fragments and derivatives thereof, such as single-chain antibodies, minibodies, Fab fragments, bi-specific antibodies and the like.

The pharmaceutical compositions that contain one or more agents can be in any form which allows for the composition to be administered to a subject. According to some embodiments, the composition will be in liquid form and the route of administration will comprise administration to a solid tissue as described herein. The term parenteral as used herein includes transcutaneous or subcutaneous injections, and intramuscular, intramedullar and intrastemal techniques.

The pharmaceutical composition is formulated so as to allow the active ingredients contained therein to be bioavailable upon administration of the composition to a subject such as a human subject. Compositions that will be administered to a subject can take the form of one or more doses or dosage units, where for example, a pre-measured fluid volume can comprise a single dosage unit, and a container of one or more compositions (e.g., drugs) in liquid form can hold a plurality of dosage units. A dose of an agent includes all or a portion of a therapeutically effective amount of a particular agent that is to be administered in a manner and over a time sufficient to attain or maintain a desired concentration range of the agent, for instance, a desired concentration range of the agent in the immediate vicinity of a delivery microdialysis probe or needle in a solid tissue, and where the absolute amount of the agent that comprises a dose will vary according to the agent, the subject, the solid tissue and other criteria with which the skilled practitioner will be familiar in view of the state of the medical and pharmaceutical and related arts. In certain embodiments, at least two doses of the agent can be administered, and in certain other embodiments 3, 4, 5, 6, 7, 8, 9, 10 or more doses can be administered.

A liquid pharmaceutical composition as used herein, whether in the form of a solution, suspension or other like form, can include one or more of the following adjuvants: sterile diluents such as water for injection, saline solution, physiological saline, Ringer's solution, saline solution (e.g., normal saline, or isotonic, hypotonic or hypertonic sodium chloride), fixed oils such as synthetic mono or digylcerides which can serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. In some embodiments, physiological saline is the adjuvant. An injectable pharmaceutical composition can be sterile. It can also be desirable to include other components in the preparation, such as delivery vehicles including but not limited to aluminum salts, water-in-oil emulsions, biodegradable oil vehicles, oil-in-water emulsions, biodegradable microcapsules, hydrogels, and liposomes.

While any suitable carrier known to those of ordinary skill in the art can be employed in the pharmaceutical compositions of this invention, the type of carrier will vary depending on the mode of administration and whether a conventional sustained drug release is also desired. For parenteral administration, such as supplemental injection of drug, the carrier can comprise water, saline, alcohol, a fat, a wax or a buffer. Biodegradable microspheres (e.g., polylactic galactide) can also be employed as carders for the pharmaceutical compositions of this invention. In some embodiments, the microsphere is larger than approximately 25 microns, while other embodiments are not so limited and contemplate other dimensions.

Pharmaceutical compositions can also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. In some embodiments, an agent (e.g., a therapeutic drug or a candidate drug) is formulated as a lyophilizate using appropriate excipient solutions (e.g., sucrose) as diluents.

Orientation

Because injected agents can be spatially defined in the tumor, tumor orientation can be important for evaluation of the agents. Loss of orientation could compromise the evaluation process because it could be unclear which injection location corresponded to which injected agent. Tumor orientation can be maintained by taking pictures of the tumor and marking the skin to show how the tumor was oriented during injection. Orientation can also be visually maintained by injection of a number of near infrared dyes. Infrared dyes can detect asymmetry of the injection array. Orientation can also be visually maintained by injection of other staining agents and inks including fluorescent tattoo inks, Henna, india ink, new methylene blue, isosulfan blue dye, and rhodamina WT. A portable light source may be used to detect tumor orientation and injected dyes during surgery.

Position Markers

Certain embodiments contemplate direct delivery of multiple agents, candidate drugs, imaging agents, positional markers, indicators of efficacy and appropriate control compositions to a plurality of spatially defined locations along parallel axes in a solid tissue, such as a solid tumor, followed, after a desired time interval, by excision of the treated tissue and evaluation or analysis of the tissue for effects of the treatments. Indicators of efficacy can be, for example, detectable indicator compounds, nanoparticles, nanostructures or other compositions that comprise a reporter molecule which provides a detectable signal indicating the physiological status of a cell, such as a vital dye (e.g., Trypan blue), a colorimetric pH indicator, a fluorescent compound that can exhibit distinct fluorescence as a function of any of a number of cellular physiological parameters (e.g., pH, intracellular Ca²⁺ or other physiologically relevant ion concentration, mitochondrial membrane potential, plasma membrane potential, etc.), an enzyme substrate, a specific oligonucleotide probe, a reporter gene, or the like. Control compositions can be, for example, negative controls that have been previously demonstrated to cause no statistically significant alteration of physiological state, such as sham injection, saline, DMSO or other vehicle or buffer control, inactive enantiomers, scrambled peptides or nucleotides, etc.; and positive controls that have been previously demonstrated to cause a statistically significant alteration of physiological state, such as an FDA-approved therapeutic compound.

In some embodiments, a pharmaceutical formulation further comprises a dye. The dye can be imaged after administration of the pharmaceutical composition to an animal tissue to observe the distribution and activity of an agent present in the same pharmaceutical composition. In some embodiments, the dye is a fluorescent dye. In some embodiments, the dye is a radioactive dye.

In some embodiments, the excised tissue can be cut into a plurality of serial histological sections along parallel planes that are substantially normal (e.g., perpendicular or deviating from perpendicular by as much as 1, 2, 3, 4, 5, 6, 7, 8, 9, 10, 11, 12, 13, 14, 15, 20, 25, 30, 35 or more degrees) to the parallel axes, for analysis by any of a number of known histological, histochemical, immunohistological, histopathologic, microscopic (including morphometric analysis and/or three-dimensional reconstruction), cytological, biochemical, pharmacological, molecular biological, immunochemical, imaging or other analytical techniques, which techniques are known to persons skilled in the relevant art. Imaging can be performed before, during or after dispenser needles are inserted into the solid tissue. Positional markers are known and include, as non-limiting examples, metal or plastic clips, fluorescent quantum dots, India ink, metal or plastic beads, dyes, stains, tumor paint or other positional markers, and can be introduced at desired positions. Markers can include any subsequently locatable source of a detectable signal, which can be a visible, optical, colorimetric, dye, enzymatic, GCMS tag, avidin, biotin, radiological (including radioactive radiolabel and radio-opaque), fluorescent or other detectable signal.

A detectable marker thus comprises a unique and readily identifiable gas chromatography/mass spectrometry (GCMS) tag molecule. Numerous such GCMS tag molecules are known to the art and can be selected for use alone or in combination as detectable identifier moieties. By way of illustration and not limitation, various different combinations of one, two or more such GCMS tags can be added to individual reservoirs of the device described herein in a manner that permits the contents of each reservoir to be identified on the basis of a unique GCMS “signature”, thereby permitting any sample that is subsequently recovered from an injection region to be traced back to its needle of origin for identification purposes. Examples of GCMS tags include α,α,α-trifluorotoluene, α-methylstyrene, o-anisidine, any of a number of distinct cocaine analogues or other GCMS tag compounds having readily identifiable GCMS signatures under defined conditions, for instance, as are available from SPEX CertiPrep Inc. (Metuchen, N.J.) or from SigmaAldrich (St. Louis, Mo.), including Supelco® products described in the Supelco® 2005 gas chromatography catalog and available from SigmaAldrich.

Biomarkers

The present disclosure exemplifies a method for evaluating changes in the physiological status of tumor cells or tumorigenic cells by measuring the biomarkers secreted by the cells. Cells may communicate and respond to physiological cues by secreting the biomarkers that can be soluble factors including autocrines, paracrines, or endocrines. Tumor cells or tumorigenic cells may secrete a plurality of biomarkers that are known in the medical arts before, during or after a change of the physiological status. The biomarkers can be proteins, peptides, amino acids, RNA, DNA, nucleic acids, proteoglycans, lipids, small organic molecules, small inorganic molecules, or ions. In some embodiments, the biomarkers can be measured in transcriptional levels as gene expressions or in protein levels. By measuring and detecting the biomarkers described herein over time, and relating the measurement to the biomarkers known in the medical art, thereby the physiological status or the changes in the physiological status of the tumor cells or tumorigenic cells, such as cell death, cell proliferation, cell signaling process or cellular responses, can be determined.

The death of tumor cells or tumorigenic cells can be via apoptosis or necrosis. Apoptosis is a process of programmed cell death, and may be activated via either the death receptor-mediated extrinsic pathway or the mitochondria-directed intrinsic pathway. Non-limiting examples of biomarkers of apoptosis that can be measured in gene expressions or protein levels include: activated caspase family such as caspases 2, 3, 7, 8, 9 and 10; tumor protein 53 (p53), phosphor-p53, p73, cyclin-dependent kinase inhibitor 1 (p21-waf1), and phosphor-H2AX/Ser 139 (pH2AX); B-cell lymphoma 2 (Bcl-2) family members such as Bcl-2, B-cell lymphoma-extra large (Bcl-XL), Bcl-xs, Bcl-W, and induced myeloid leukemia cell differentiation protein (Mc1-1); pro-apoptotic protein family such as Bcl-2-associated X protein (Bax), and Bcl-2 homologous antagonist/killer (Bak); Bcl-2 homology (BH) domain family such as BH1, BH2, BH3, BH4, Bcl-2-associated death promoter (Bad), p53 upregulated modulator of apoptosis (PUMA), NOXA, Bcl-2 modifying factor (Bmf), Bcl-2 interacting killer (Bik), Bcl-2-related ovarian killer (Bok), Bcl-2 interacting mediator of cell death (Bim), and BH3 interacting-domain death agonist (Bid); modulators of apoptosis proteins such as apoptotic protease activating factor 1 (APAF-1), apoptosis inducing factor (AIF), inhibitors of apoptosis (IAP) such as cIAP1, cIAP2, Cp-IAP, Op-IAP, XIAP, NAIP, survivin, and second mitochondria-derived activator of caspases (SMAC); markers to measure extent of DNA oxidative damage such as 8-hydroxy-2-deoxyguanosine and 3-nitrotyrosine; other biomarkers related to apoptosis such as cytochrome c, N-hydroxy-L-arginine (NOHA), 14-3-3 protein, tumor necrosis factor (TNF)-related apoptosis inducing ligand (TRAIL), reactive oxygen species (ROS), externalized phosphatidylserine, cytokeratins, poly(ADP-ribose) polymerase, nucleosomal DNA, apoptosis antigen 1 (Apo-1), TNF receptor superfamily, member 6 (Fas), Fas ligand (FasL), Fas-associated death domain protein (FADD), phosphorylated-FADD, glutathione-S-transferase-isoenzyme π (Gst-π), β-galactosidase, phosphorylated retinoblastoma suppressor protein and the like.

Necrosis is a premature death of cells or tissues, and may be caused by factors external to the cells or tissues. Other physiological events such as inflammatory responses of the cells may be triggered with necrosis. Non-limiting examples of biomarkers related to necrosis of tumor cells or tumorigenic cells that can be measured in gene expressions or protein levels include tumor necrosis factor (TNF), cachexin, cachectin, lymphotoxin, cyclophilin A, interleukin-1, 2, 3, 4, 5, 6, 7, 8, 9, 10, 11, 12, 13, 14, 15, 16 and 17, alpha1-antitrypsin, copeptin, myeloperoxidase, FLICE-like inhibitory protein (FLIP), transducer and activator of transcription (STAT), tumor necrosis factor receptor superfamily, member 19 (TROY), cyclooxygenase (COX)-1, COX-2, cell death factors, macrophage inflammatory proteins, macrophage activating factors, macrophage migration inhibitory factors, neuroleukin, immunologic suppressor factors, transfer factors, oncostatin, osteopontin, interferon type I, interferon gamma, interleukin 1 receptor antagonist protein, CD70, CD30, CD40, 4-1BB ligand, ectodysplasins, B-cell activating factor, receptor activator of nuclear factor kappa-B ligand (RANKL), lymphotoxin and the like.

In addition to measuring the biomarkers that can be related to cell death, the current disclosure further provides a method to measure biomarkers that can be measured in gene expressions or protein levels to relate to the proliferation/growth or mitotic activities of tumor cells or tumorigenic cells. Non-limiting examples of biomarkers described herein include Akt protein kinase B, Wilms tumor marker, retinoblastoma (Rb), Ki-67, proliferating cell nuclear antigen (PCNA), serine/threonine kinase, mammalian target of rapamycin (mTOR), neurotrophin, protein Mis18 beta, myostatin, cyclin dependent kinases (Cdk) 1, 2, 4, and 6, cyclin dependent kinase comples 2 (Cdc2 p34), cyclin D1, cyclin D2, cyclin D3, cyclin E, cyclin A, growth differentiation factors 1, 2, 3, 5, 6, 9, 10 and 15 and the like.

The physiological status of a cell may be heavily modulated by a plurality of signal transduction pathways. Signal transduction occurs when an extracellular signaling molecule or a ligand binds to and further activates a cell surface receptor, thereby altering intracellular molecules creating a response. In some preferred aspects, the biomarkers related to signal transduction changes of tumor cells or tumorigenic cells can be measured in gene expressions or protein levels. The biomarkers described herein can participate in the signaling pathways as growth factors, enzymes, signaling factors, ligands, intermediate molecules generated in biological pathways, hormones, nutrients, transmembrane proteins, extracellular matrix proteins, intracellular components, downstream factors of protein phosphorylation and the like. Non-limiting examples of signal transduction biomarkers include human epidermal growth factor receptor (HER) family molecules such as HER1, 3, and 4; phosphatidylinositol 3-kinases (PI3K)/protein kinase B (Akt) signaling pathway molecules as PI3K/AKT, microtubule-associated protein kinase (MAPK)/extracellular signal-regulated kinase (ERK) pathway molecules such as MAPK, mitogen-activated protein kinase (MEK), Ras, proto-oncogene serine/threonine-protein kinase (RAF), ERK1 and 2; hedgehog pathway proteins such as sonic hedgehog, desert hedgehog, indian hedgehog, hedgehog-interacting protein, smoothened protein (SMO), Gli-1, Gli-2, Gli-3, and forkhead box O (FoxO)-1; Wnt signal transduction pathway modulators such as Wnt1, 2, 2B, 3, 3A, 4, 5A, 5B, 6, 7A, 7B, 8A, 8B, 9A, 9B, 10A, 10B, 11, 16, Wnt1-inducible-signaling pathway protein 1 (Wisp-1), Wisp-2, and β-catenin; parathyroid hormone-related proteins such as hypercalcemic hormone of malignancy, parathyroid hormone like tumor factor; phosphatase and tensin homolog (PTEN), serine/threonine-protein kinase (SGK3), eukaryotic translation initiation factor 4E-binding protein 1 (4E-BP1), tymidine kinase, growth hormone, pyruvate dehydrogenase lipoamide kinase isozyme 1 (PDK1), citrate, nitride oxide, P70S6 kinase, glycogen synthase kinase 3 (GSK-3), Src homology 2 domain containing (SHC)-transforming protein 1, CD117, platelet-derived growth factor receptor (PDGFR)-α, PDGFR-β, vascular endothelial growth factor receptor-2 (VEGFR-2), epidermal growth factor receptor (EGFR), matrix metalloproteinase (MMP)-1, CD9, keratin 7, p2′7, parafibromin, BMI1 polycomb ring finger oncogene (Bmi-1), 14-3-3σ, cystatin-SA, epididymal secretory protein E4, whey acidic protein (WAP) four-disulfide core domain protein 2 (WFDC2), adiponectin, leptin, resistin, agouti signaling protein, agouti-related protein, angiopoietins, angiostatic proteins, cysteine-rich protein 61, nephroblastoma overexpressed protein, peptide PHI, peptide YY, insulin, glucose, pituitary hormones, placental hormones, relaxin, secretin, urocortins, urotensins, vasoactive intestinal peptide, autocrine motility factor, beta-thromboglobulin, leukemia inhibitory factor, leukocyte migration-inhibitory factors, lymphotoxin-alpha, endothelin, enphrin, bradykinin, kininogens, tachykinins, chemokines such as chemokine C, CC, CXC, CX3C and the like.

In certain aspects, the biomarkers capable of triggering a signal transduction pathway, in turn altering a cellular response can be a growth factor. Non-limiting examples of growth factors that can be measured in gene expressions or protein levels to relate tumor cells or tumorigenic cells to a physiological status include erythropoietin (EPO), angiopoietin (Ang), stem cell factor (SCF), vascular endothelial growth factor (VEGF), fibroblast growth factor (FGF), nerve growth factor (NGF), hematopoietic cell growth factor, hepatocyte growth factor, hepatoma-derived growth factor, migration-stimulating factor, autocrine motility factor, epidermal growth factor (EGF), insulin-like growth factor 1 (IGF-1), transforming growth factor (TGF), cartilage growth factor (CGF), keratinocyte growth factor (KGF), skeletal growth factor (SGF), osteoblast-derived growth factor (BDGF), cytoline growth factor (CGF), colony stimulating factor (CSF), integrin modulating factor (IMF), platelet-derived growth factor (PDGF), calmodulin, bone morphogenic proteins (BMP), tissue inhibitor matrix metalloproteinase (TIMP), and the like.

In certain embodiments, the biomarkers are immunohistochemistry (IHC) markers. Non-limiting examples of IHC markers that can be measured include hematopoetic markers, breast markers, carcinoma or mesothelial markers, colon markers, central nervous system markers, infectious disease markers, keratin or epithelial markers, lung markers, melanocytic markers, neuroendocrine markers/other hormones, other organ-related markers, prognostic other markers, prostate markers, stromal markers or tumor markers. Hematopoetic markers include, but not limited to: annexin A1, BCL2 follicular lymphoma marker, BCL6 follicle center B cell marker, CD10, CD20, CD23, CD79a, cyclin D1, hairy cell leukemia marker, multiple myeloma oncogene 1, PAX-g B cell transcriptional factor, ZAP 70, CD34, CD68, CD99, CD117, glycophorin-A, myeloperoxidase, terminal deoxynucleotidyl transferase, von willebrand factor VIII, anaplastic lymphoma kinase-1, CD15, CD30, fascin, CD45, CD138, kappa immunoglobulin light chains, lambda immunoglobulin light chains, plasma cell p63, CD1a, CD2, CD3, CD4, CD5, CD7, CD8, CD43, CD56, CD57 and granzyme B. Breast markers include, but not limited to: Akt protein kinase, cytokeratin 5, p63, epithelial antigen, cathepsin D, cytokeratin 8, HMW cytokeratin high molecule weight, cytokeratin 5/6, cytokeratin 7, cytokeratin 19, cytokeratin 20, E-cadherin, estrogen receptor, HER2/neu, Ki67 cell proliferation marker, p53 tumor suppressor gene protein, progesterone receptor and smooth muscle actin. Carcinoma or medothelial markers include, but not limited to: BER-EP4 epithelial antigen, calretinin, ERA epithelial related antigen, cervical or gynecological markers, p16 tumor suppressor gene protein, ProEx C biomarker, TAG72 and wilms tumor marker. Colon markers include, but not limited to: epidermal growth factor receptor, CDX2, microsatellite instability marker such as MLH1, MSH2, MSH6, PMS2 and p53. CNS markers include, but not limited to: human glial fibrillary acidic protein and neurofilament. Infectious disease markers include, but not limited to: cytomegalovirus, herpes simplex virus type I, II, pylori H and varicella zoster virus. Keratin and epithelian markers include, but not limited to: cytokeratin 5/6, cytokeratin 7, cytokeratin 8/18, cytokeratin 19, cytokeratin 20, cytokeratin high molecular weight, caldesmon smooth muscle, p63, collagen 9, smooth muscle myosin, cytokeratin cocktail and epithelial membrane antigen. Lung markers include, but not limited to: 34BE12, HMW cytokeratin high molecular weight, excision repair cross complementing polypeptide, synaptophysin and thyroid transcription factor-1. Melanocytic markers include, but not limited to: HMB melanoma associated marker 45, melanoma cocktail, melanoma associated marker 1, s100 protein and tyrosinase. Neuroendocrine markers and other hormones include, but not limited to: androgen receptor, calcitonin, chromogranin A, G cell antral pyloric mucosa, neuron-specific enolase, somatostatin and synaptophysin. Other organ-related markers include, but not limited to: CEA carcinoembryonic antigen, calectin-3, gross cyctic disease fluid protein 15, hepatocyte antigen, adrenal cortical inhibin and renal cell carcinoma marker. Prostate markers include, but not limited to: PIN2 cocktail, PIN4 cocktail, prostate specific antigen, prostatic acid phosphorase and p504s gene product. Stromal markers include, but not limited to: CD31, podoplanin, DOG1 derived from GIST1, desmin filament protein, factor XIIIa fibrohistocytic, human herpesvirus type 8, muscle specific actin, myogenin muscle marker, myoglobin cardiac and skeletal marker, s100 protein, smooth muscle actin, smooth muscle myosin and vimentin. Tumor markers indluce, but not limited to: alpha detoprotein, Ca 19-9 CI, Ca-125 epitheliod malign marker and survivin.

In some embodiments, the biomarkers that can be measured in gene expressions or protein levels are metabolites or metabolic biomarkers. Non-limiting examples of metabolites or metabolic biomarkers include: adenosine triphosphate (ATP), adenosine diphosphate (ADP), adenosine monophosphate (AMP), cyclic adenosine monophosphate (cAMP), Guanosine-5′-triphosphate (GTP), Guanosine-5′-diphosphate (GDP), Guanosine-5′-monophosphate (GMP), nicotinamide adenine dinucleotide phosphate (NADP), NADPH, nicotinamide adenine dinucleotide (NAD), NADH, proliferating cell nuclear antigen, glucose, glucose-6-phosphate, fructose-6-phosphate, fructose 1,6-b phosphate, ribose-5-phosphate, erythrose-4-phosphate, xylulose 5-phosphate, glyceraldehyde-3-phosphate, sedoheptulose 7-phosphate, 3 ribulose-5-phosphate, 1 ribose-5-phosphate, phosphoenolpyruvate, 2-phosphoglycerate, 3-phosphoglycerate, 1,3-phosphoglycerate, dihydroxyacetone phosphate, malate, oxaloacetate, ketoglutarate, lactate, glutamine, alanine, glutamate, pyruvate, fatty acids, acetyl-coA, citrate, glycerol, uric acid, cholesterols, eicosanoids, glycolipids, phospholipids, shpingolipids, steoid, triacylglycerols, albumin, insulin, diols, Ros, NO, bilirubin, phosphor-creatine, ketone bodies, L-ornithine, argininosuccinate, fumarate, L-arginine, urea, carbamoyl phosphate, ornithine, citrulline, histidine, isoleucine, leucine, lysine, methionine, phenylanine, threonine, tryptophan, valine, asparagines, aspartic acid, cysteine, glutamic acid, glycine, proline, selenocysteine, serine, taurine, tyrosine, citric acid and the like.

In some embodiments, the biomarkers could be ions. Non-limiting examples include hydrogen, potassium, sodium, calcium, chloride, magnesium, bicarbonate, phosphate, hydroxyl, iodine, copper, iron, zinc, sulfate and the like.

The agent may comprise a plurality of agents. The plurality of agents may comprise at least one of a negative control composition and a positive control composition. The plurality of agents may comprise at least one position marker. At least one of the plurality of agents may be a candidate effective agent. At least one of the plurality of agents may comprise an indicator of efficacy, which in certain further embodiments can comprise at least one of a nanoparticle, a nanostructure, and an indicator dye. At least one of the agents may be selected based on a clinically demonstrated efficacy of the respective agent. The methods of the disclosure may comprise assessing, with respect to at least one of the plurality of agents, at least one of efficacy, activity, and toxicity of the agent.

Target Tissues

In some embodiments, the present disclosure exemplifies a method for screening agents in a solid tissue. Solid tissues are well known to the medical arts and may include any cohesive, spatially discrete non-fluid defined anatomic compartment that is substantially the product of multicellular, intercellular, tissue and/or organ architecture, such as a three-dimensionally defined compartment that may comprise or derive its structural integrity from associated connective tissue and may be separated from other body areas by a thin membrane (e.g., meningeal membrane, pericardial membrane, pleural membrane, mucosal membrane, basement membrane, omentum, organ-encapsulating membrane, or the like). Non-limiting exemplary solid tissues may include brain, liver, lung, kidney, prostate, ovary, spleen, lymph node (including tonsil), thyroid, pancreas, heart, skeletal muscle, intestine, larynx, esophagus and stomach. Anatomical locations, morphological properties, histological characterization, and invasive and/or non-invasive access to these and other solid tissues are all well known to those familiar with the relevant arts. In some embodiments, the tissue is, or is suspected of being, cancerous, inflamed, infected, atrophied, numb, in seizure, or coagulated. In some embodiments, the tissue is, or is suspected of being, cancerous. In some embodiments, the tissue is cancerous.

In some embodiment, the present method is directed to cancer, and the target tissue comprises a tumor, which may be benign or malignant, and comprises at least one cancer cell selected from the group consisting of a prostate cancer cell, a breast cancer cell, a colon cancer cell, a lung cancer cell, a brain cancer cell, and an ovarian cancer cell. In certain embodiments, the tumor comprises a cancer selected from lymphoma, adenoma, adenocarcinoma, squamous cell carcinoma, basal cell carcinoma, small cell carcinoma, large cell undifferentiated carcinoma, chondrosarcoma and fibrosarcoma. In certain embodiments the selected region of tissue is a portion of a tumor in a subject, and in certain further embodiments the subject is one of a preclinical model OR a human patient.

Certain preferred embodiments contemplate a subject or biological source that is a human subject such as a patient that has been diagnosed as having or being at risk for developing or acquiring cancer. Certain embodiments contemplate a human subject that is known to be free of a risk for having, developing or acquiring cancer by such criteria.

Certain other embodiments contemplate a non-human subject or biological source, for example a non-human primate such as a macaque, chimpanzee, gorilla, vervet, orangutan, baboon or other non-human primate, including such non-human subjects that may be known to the art as preclinical models, including preclinical models for solid tumors and/or other cancers. Certain other embodiments contemplate a non-human subject that is a mammal, for example, a mouse, rat, rabbit, pig, sheep, horse, bovine, goat, gerbil, hamster, guinea pig or other mammal; many such mammals may be subjects that are known to the art as preclinical models for certain diseases or disorders, including solid tumors and/or other cancers. The range of embodiments is not intended to be so limited, however, such that there are also contemplated other embodiments in which the subject or biological source may be a non-mammalian vertebrate, for example, another higher vertebrate, or an avian, amphibian or reptilian species, or another subject or biological source. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells). In certain embodiments of the present invention, the tissue of a transgenic animal may be targeted.

Methods of the current invention are suitable for administering agents to a variety of animal tissues; thus the methods have medical and veterinary uses. In some embodiments, the animal tissue is soft tissue. Non-limiting examples of soft tissue include muscle, adipose, skin, tendons, ligaments, blood, and nervous tissue. In some embodiments, the animal is a reptile, an amphibian, an ayes, or a mammal. In some embodiments, the animal is a mammal. In some embodiments, the animal is a mouse. In some embodiments, the animal is a human. In some embodiments, the animal is a pet, a companion, a guardian, a working animal, a breeding animal, a service animal, a racing animal, a farm animal, a herded animal, or a laboratory animal.

In some embodiments, the target tissue does not exhibit features of a disease, but may be used to assess the response of an individual tissue to one or more compounds. In some cases, one or more compounds may be administered to produce an altered physiologic state within a tissue. An altered physiologic state can be any detectable parameter that directly relates to a condition, process, pathway, dynamic structure, state or other activity in a solid tissue (and in some embodiments in a solid tumor) including in a region or a biological sample that permits detection of an altered (e.g., measurably changed in a statistically significant manner relative to an appropriate control) structure or function in a biological sample from a subject or biological source. The methods of the present invention thus pertain in part to such correlation where an indicator of altered physiologic state can be, for example, a cellular or biochemical activity, including as further non-limiting examples, cell viability, cell proliferation, apoptosis, cellular resistance to anti-growth signals, cell motility, cellular expression or elaboration of connective tissue-degrading enzymes, cellular recruitment of angiogenesis, or other criteria as provided herein.

The tissue may be a tumor. In some embodiments, the tumor is resistant to a therapy, for example, a chemotherapy. The tumor may respond to the therapy initially but develop resistance suddenly or gradually. There may be a variety of reasons for the development of drug resistance, including: (1) Cell mutation. Cells that are not killed by the chemotherapy may mutate and become resistant to the drug. Their multiplication may produce more resistant cells than cells that are sensitive to the chemotherapy; (2) Gene amplification. Cancer cells may produce hundreds of copies of a particular gene. This gene may trigger an overproduction of protein that render the anticancer drug ineffective; (3) P-gp mediated efflux. Cancer cells may pump the drug out of the cell using a molecule called p-glycoprotein; and (4) Transporter inhibition. Cancer cells may stop taking in the drugs because the protein that transports the drug across the cell wall stops working.

Altered physiologic state can further refer to any condition or function where any structure or activity that is directly or indirectly related to a solid tissue function has been changed in a statistically significant manner relative to a control or standard, and can have its origin in direct or indirect interactions between a solid tissue constituent and an introduced agent, or in structural or functional changes that occur as the result of interactions between intermediates that can be formed as the result of such interactions, including metabolites, catabolites, substrates, precursors, cofactors and the like. Additionally, altered physiologic state can include altered signal transduction, respiratory, metabolic, genetic, biosynthetic or other biochemical or biophysical activity in some or all cells or tissues of a subject or biological source, in some embodiments in some or all cells of a solid tissue, and in some embodiments in some or all cells of a tumor such as a solid tumor in a solid tissue. As non-limiting examples, altered biological signal transduction, cell viability, cell proliferation, apoptosis, cellular resistance to anti-growth signals, cell motility, cellular expression or elaboration of connective tissue-degrading enzymes, cellular recruitment of angiogenesis, or other criteria including induction of apoptotic pathways and formation of atypical chemical and biochemical crosslinked species within a cell, whether by enzymatic or non-enzymatic mechanisms, can all be regarded as indicative of altered physiologic state.

Methods

There can be provided a method for identifying relative efficacies of a plurality of agents for treating a subject, comprising injecting each of a plurality of candidate effective agents into a respective location in an injection site in a solid tissue in a subject; excising from the subject at least the injection site of the solid tissue; and evaluating the excised injection site for an altered physiologic state at each of the respective locations, and identifying relative efficacies of the plurality of agents. The excising may comprise one of excising at least 48 hours after the injecting, excising at least 72 hours after the injecting, excising 72 to 96 hours after the injecting, and excising at least one week after the injecting.

The disclosure provides for methods for systemic delivery of a plurality of agents in conjunction with microdosing a solid tissue using the device of the disclosure. In some instances, system delivery of a plurality of agents can be performed in an organism. An organism can be a subject or a patient. An organism can be a mammal (e.g., human, a rat, a cow, a dog, a mouse, a cat, a horse, a non-human primate (e.g., chimpanzee, monkey, ape), a vertebrate (e.g., reptiles, fish, birds, amphibians), or an invertebrate (e.g., mollusk, echinoderm, arachnids, insects, crustacean). The organism can be a pet, a companion, a guardian, a working animal, a breeding animal, a service animal, a racing animal, a farm animal, a herded animal, or a laboratory animal.

In some embodiments, an agent (or plurality of agents) is introduced into an organism systemically followed by local microdosing of an agent (or plurality of agents) in a solid tissue. In some instances, the local microdosing of the solid tissue is performed before the systemic dosing. In some instances, the local microdosing of the solid tissue is performed at the same time as the systemic dosing. The systemically dosed agent and the local microdosed agent can be the same. The systemically dosed agent and the local microdosed agent can be different. Some of the systemically dosed agents can be the same as the local microdosed agents and some can be different. The systemic agents and microdosed agents can act synergistically or antagonistically.

Also provided herein is a method of determining efficacy of a cancer treatment regimen, comprising simultaneously introducing an agent to a plurality of positions in a solid tumor in a subject in vivo; removing the tumor from the subject; and evaluating an effect of the agent on the tumor in vitro. The agent may comprise a plurality of agents and the introducing may comprise distributing each of the plurality of agents to a respective one of the plurality of positions in the tumor. In another embodiment there is provided a method, comprising introducing an agent to a region of solid tissue in a subject by distributing the agent to a plurality of positions along an axis within the region of solid tissue in vivo; removing the region of solid tissue from the subject; and evaluating an effect of the agent on the region of solid tissue in vitro. In a further embodiment the region of solid tissue comprises a tumor.

The axis may be one of a plurality of parallel axes in the region of solid tissue, and the introducing may comprise distributing the agent along each of the plurality of parallel axes. The introducing may comprise simultaneously distributing the agent along each of the plurality of parallel axes, and the plurality of parallel axes may be arranged in an array. The method may comprise introducing at least two position markers to the region of solid tissue along a respective one of the plurality of parallel axes, and the introducing at least two position markers may comprise distributing the at least two position markers along respective parallel axes within the region of solid tissue. The at least two position markers each may comprise a detectable label that is selected from the group consisting of a radiolabel, a radio-opaque label, a fluorescent label, a colorimetric label, a dye, an enzymatic label, a GCMS tag, avidin, and biotin.

In certain other embodiments of the method, the agent may be one of a plurality of agents and the axis may be one of a plurality of parallel axes arranged in an array in the region of solid tissue, and wherein the introducing can comprise distributing each of the plurality of agents to a plurality of positions along a respective one of the plurality of parallel axes. The method may comprise imaging the solid tissue prior to the introducing, imaging the solid tissue concurrently with the introducing, and imaging the solid tissue after the introducing. The evaluating may comprise sectioning the region of solid tissue into a plurality of sections normal to the parallel axes. The evaluating may comprise detecting within the solid tissue an altered physiologic state that results from at least one of the plurality of agents. The detecting may comprise, with respect to the at least one of the plurality of agents, at least one of detecting a degree of permeation of the agent through the solid tissue, detecting a physicochemical effect of the agent on the tissue, and detecting a pharmacological effect of the agent on the tissue. The evaluating may comprise determining the effects of at least two of the plurality of agents on a same position within the region of the solid tissue. The evaluating may comprise determining the effects of at least two of the plurality of agents on adjacent positions within the region of the solid tissue.

The evaluating may comprise differentiating a degree of the effect of at least one of the plurality of agents on different sections of the solid tissue according to different characteristics of the different sections of the solid tissue. The evaluating may comprise comparing a first effect of at least a first one of the plurality of agents on the solid tissue with a second effect of at least a second one of the plurality of agents on the solid tissue. The evaluating may comprise, with respect to at least one of the plurality of agents, assessing at least one of efficacy, activity, and toxicity on the region of solid tissue. In certain other embodiments the method comprises deselecting at least one of the plurality of agents based on the evaluating. In certain other embodiments the method comprises selecting at least one of the agents based on the evaluating. In certain other embodiments the method comprises prioritizing at least two of the plurality of agents based on the evaluating. In certain other embodiments the method comprises distributing the plurality of agents to a plurality of positions, each along a respective one of a plurality of parallel axes within a region of solid tissue within each of a plurality of subjects. In certain further embodiments the method comprises one of (i) selecting at least one of the plurality of agents based on the evaluating, (ii) deselecting at least one of the plurality of agents based on the evaluating, and (iii) prioritizing at least two of the plurality of agents based on the evaluating. In certain other embodiments the method comprises one of (i) selecting at least one of the plurality of subjects based on the evaluating, (ii) deselecting at least one of the plurality of subjects based on the evaluating, and (iii) prioritizing at least two of the plurality of subjects based on the evaluating. In certain other embodiments the evaluating comprises determining a level of altered physiologic state of the solid tissue near at least one of the plurality of parallel axes.

In certain embodiments there is provided a method of screening subjects for eligibility to participate in a clinical trial of one or more agents, comprising (a) introducing one or more agents to a region of solid tissue in one or more subjects in vivo by distributing each of said agents to a plurality of positions along an axis within the region in each subject; (b) removing the region of solid tissue from each of said subjects; and (c) evaluating each region removed in (b) for an effect of each agent on the respective position along the axis within the region, wherein either (i) for any given agent or agents presence of a detectable effect of said agent or agents on the solid tissue region from the subject indicates eligibility of the subject for participation in a clinical trial of the agent or agents, (ii) for any given agent or agents absence of a detectable effect of said agent or agents on the solid tissue region from the subject indicates ineligibility of the subject for participation in a clinical trial of the agent or agents, or (iii) both (i) and (ii).

In certain embodiments there is provided a method of rating a candidate agent for development into a therapeutic agent for treating a solid tumor, comprising (a) introducing one or more candidate agents to a region of a solid tumor of known tumor type in each one or more subjects having a tumor of the known tumor type, by distributing each of said candidate agents to a plurality of positions along an axis within the region in each subject; (b) removing the region of solid tumor from each of said subjects; and (c) comparing each region removed in (b) for an effect of each candidate agent on the respective position along the axis within the region, wherein an agent that results in a greater beneficial effect when introduced to the tumor receives a higher rating for development into a therapeutic agent for treating the solid tumor, and an agent that results in a lesser beneficial effect when introduced to the tumor receives a lower rating for development into a therapeutic agent for treating the solid tumor.

The present invention provides devices and methods that are useful for the classification and/or stratification of a subject or subject population, including for use in drug discovery and in pharmacogenomics. In these and related embodiments, correlation of one or more indicia of an altered physiological state with a position at which a given candidate agent has been introduced in a solid tumor can be used to gauge the subject's responsiveness to, or the potential efficacy of, a particular therapeutic treatment; related embodiments contemplate this approach for “deselection”, or elimination from consideration as potential therapies, of candidate agents in which no evidence of an altered physiological state is detected at a site of introducing in the tumor.

As described herein, determination of levels of at least one indicator of altered physiologic state can also be used to stratify a subject population for eligibility to participate in a clinical trial. These and related embodiments are contemplated as usefully providing advantages associated with evaluation of candidate therapeutic compounds at an earlier stage of development than is currently the case. For instance, it is not currently standard clinical trial practice to establish biomarker parameters (which can be the basis for exclusion of subjects) prior to Phase III studies, whereas the embodiments described herein can provide useful results even in the absence of established biomarker criteria, for example, at Phase II. Accordingly it is envisioned that through the practice of certain presently disclosed embodiments, relevant information on the properties of a candidate agent can be obtained earlier in a solid tumor oncology drug development program than has previously been the case, including in a manner which can time-efficiently and cost-effectively permit elimination from a clinical trial of subjects for whom no response or benefit can be expected based on a nonresponder result for a particular candidate agent.

For example, stratification of a subject population according to levels of at least one indicator of altered physiologic state, determined as described herein, can provide a useful marker with which to correlate the efficacy of any agent being used in cancer subjects, and/or to classify subjects as responders, nonresponders or possible responders.

Detection

In some embodiments it is contemplated that the target region in a solid tissue can be imaged using known techniques to evaluate the effects of the agents. The imaging can be performed by any suitable process or method, including, for example, radiographic imaging, magnetic resonance imaging, positron emission tomogoraphy, biophotonic imaging, etc. In some embodiments, the target region can be imaged repeatedly before, during, and after the delivery process.

Upon imaging, the level of the reporting signal can be quantified. Observation and/or quantification of the reporting signal can be used to make informed research and health care decisions regarding the use and efficacy of an agent. Non-limiting examples of decisions that can be made on such observations include fluid volume quality control, positional tracking, and drug biodistribution. Such experiments can be performed on a lower mammal, for example, a mouse, to provide reporting signals that can be used to make informed predictions regarding the activity of a potential agent in a human. Animal studies of this type can be used to avoid the inherent uncertainty and inaccuracies that arise by conducting drug efficacy studies in cells in controlled environments instead of in the native environment.

Fluorescence signals can quantified. Fluorescence signals can be compared with a standard or a control to determine up-regulation or down-regulation of a biological pathway. Such observations can be used to make predictions regarding the therapeutic value of drug candidates.

Certain embodiments relate to introducing an agent into a solid tissue in a subject, and/or excising all or a portion of a solid tissue from a subject, and/or obtaining one or more biological samples from a solid tissue that can be in a subject, and/or screening one or more subjects for clinical trial eligibility, and/or any number of other methods that can involve a subject, which includes a subject or biological source.

The subject or biological source can be a human or non-human animal, a transgenic or cloned or tissue-engineered (including through the use of stem cells) organism, a primary cell culture or culture adapted cell line including but not limited to genetically engineered cell lines that can contain chromosomally integrated or episomal recombinant nucleic acid sequences, immortalized or immortalizable cell lines, somatic cell hybrid cell lines, differentiated or differentiatable cell lines, transformed cell lines and the like. The subject or biological source can be suspected of having or being at risk for having a malignant condition. The subject or biological source can be known to be free of a risk or presence of such disease.

Some embodiments may relate to a method for selective delivery of a fluid-phase agent to a solid tissue. As also noted above, such selective delivery may obviate the need for excessive systemic concentrations of therapeutic or candidate agents in order to achieve therapeutically effective concentrations in the desired solid tissue, thereby avoiding clinically detrimental toxicities to uninvolved tissues and also avoiding undesirable side-effects. Related embodiments contemplate the testing of currently non-approved candidate agents through such selective delivery to a solid tissue. Without wishing to be bound by theory, according to these embodiments, direct effects of the candidate agent on the solid tissue (e.g., solid tumor) can be evaluated by in vivo administration followed by ex vivo analysis of excised tissue, without threatening the health of the subject, because the dose used for direct administration into the solid tissue can be far lower than the minimal dose that would otherwise be administered systemically. (The minimal dose can be the smallest amount of the agent that will produce a desired physiologic effect in the subject.) The agent that can be selectively administered to the solid tissue according to the present disclosure can be either undetectable outside the solid tissue, or if detectable outside the solid tissue, the agent can be present at less (in a statistically significant manner) than the minimal dose.

Such considerations pertain in related embodiments, wherein detection in a solid tissue of an altered physiologic state subsequent to introducing an agent or a plurality of agents may include detecting a degree of permeation of the agent(s) through the solid tissue, detecting a degree of absorption of the agent(s) in the tissue, detecting a physicochemical effect of the agent(s) on the tissue, and/or detecting a pharmacological effect of the agent(s) on the tissue. Assays, including fluorescence assays, of drug permeation or penetration in solid tissues can be configured further according to the present disclosure, for instance, through the detection in histological serial sections of a detectable label that has been co-administered to the solid tissue, prior to excision and sectioning, with an agent of interest.

In such embodiments, permeation or penetration may refer to the area of retention of an agent in the solid tissue in the immediate vicinity of the needle from which the agent was introduced exclusive of perfusion (entry into and dispersion via any blood vessel), and can include retention of the agent in extracellular space or extracellular matrix or in association with a cell membrane or intracellularly. Permeation can be distinct from a physicochemical effect, which may refer to microscopically detectable mechanical disruption of tissue that results from the needle insertion or fluid injection itself, or from non-biological mechanical or chemical tissue disruption caused by the agent (e.g., damage to cell membranes or disintegration of cell-cell junctions). Pharmacological effects may include statistically significant alterations of a cell or tissue physiological state that are detectable as consequences of the molecular mechanism of action of the agent, for example, cytoskeletal reorganization, extension or withdrawal of cellular processes, or evidence of biological signal transduction as can be detected using any of a number of known cytological, biochemical, molecular biological or other read-outs. Comparison of serial sections can permit distinguishing the nature of the effect that is detected histologically.

The solid tissue may comprise a tumor, wherein agent delivery can be made to, and/or sample retrieval can be made from, the solid tumor. A selected region of a tumor can comprise the site into which the needles of the presently described devices are inserted, introduced or otherwise contacted with the tumor. The region can be selected on any number of bases, including based on imaging that can be conducted before, during or after a step of needle insertion, introduction or contacting, or based on imaging conducted before, during or after excising the solid tissue from a subject, or based on other criteria including but not limited to anatomic location, accessibility in the course of a surgical procedure, degree of vascularization or other criteria.

Solid tumors may be of any type. The solid tumor can be a benign tumor or a malignant tumor, which can further be a primary tumor, an invasive tumor or a metastatic tumor. A solid tumor can comprise a prostate cancer cell, a breast cancer cell, a colon cancer cell, a lung cancer cell, a brain cancer cell and an ovarian cancer cell, but the invention is not intended to be so limited and other solid tumor types and cancer cell types can be used. For example, the tumor can comprise a cancer selected from adenoma, adenocarcinoma, squamous cell carcinoma, basal cell carcinoma, small cell carcinoma, large cell undifferentiated carcinoma, chondrosarcoma and fibrosarcoma, or the like.

According to certain presently contemplated embodiments, the efficacy of an agent can be identified by detecting an altered physiologic state as provided herein, including by assessing any of a number of biological parameters characteristic of a cancer cell. Such biological parameters can include, determining the effect of a candidate agent on one or more traits exhibited by cancer cells, determining one or more of (i) an ability to evade apoptosis, (ii) acquisition of self-sufficiency in growth signals, (iii) insensitivity to growth-inhibitory signals, (iv) acquisition of tissue invasive and metastatic phenotype, (v) unlimited replicative potential, and (vi) sustained angiogenesis. Multiple approaches for detecting the presence of these alterations of physiologic state can be used, and can be adapted to a particular excised tumor system. Non-limiting examples of parameters that can be assayed to identify an altered physiologic state include assays of cell viability, cell division, apoptosis, necrosis, cell surface marker expression, cellular activation state, cellular elaboration of extracellular matrix (ECM) components or of ECM-degrading enzymes, morphometric analysis, extension or retraction of cellular processes, cytoskeletal reorganization, altered gene expression, e.g., by in situ hybridization of immunohistochemistry, and the like.

Mass Spectrometry

The present invention provides methods for multiplexed evaluation of agents with mass spectrometry. Common mass spectrometer configurations and techniques include matrix-assisted laser desorption/ionization source with a time-of-flight mass analyzer (MALDI-TOF), inductively coupled plasma-mass spectrometry (ICP-MS), accelerator mass spectrometry (AMS), thermal ionization-mass spectrometry (TIMS) and spark source mass spectrometry (SSMS). In some embodiments, a sample is analyzed with MALDI Imaging Mass Spectrometry.

For the evaluation by mass spectrometry, several protocols may be used to prepare tissue samples. In some embodiments, a solid tissue may be removed from a subject and frozen at a low temperature. The tissue can then be histologically sectioned. The plane of the section may be about orthogonal to the axis of agent delivery. In some other embodiments, individual cells or cluster of cells are isolated by laser-capture microdissection or contact blotting of a tissue on a membrane target. In some other embodiment, tissue sample may be obtained by biopsy.

The tissue sample may be mounted to a plate, such as a stainless steel plate. A solution of matrix may be coated to the sample. The solution of matrix may aid the ionization of various of molecules in the tissue sample. The solution may comprise an organic acid, for example, sinapinic acid. The sample may be dried prior to analysis by mass spectrometry. To carry out the Mass Spectrometry analysis, a raster with consecutive laser spot may be irradiated on the surface of tissue. The laser spot may have a diameter in a range of about 10-100 μm. In some embodiments, the diameter is about 25 μm. The laser position may be fixed while the sample plate may be repositioned to obtained a mass image of the tissue sample.

After the irradiation, the atomic mass window of interest may be analyzed to determine the spatial arrangements of specific biomarkers. The spatial arrangements of specific biomarkers may be graphically depicted by plotting mass of the biomarkers along X and Y axes of the sample. Furthermore, mass of the biomarkers may be graphically depicted along Z axis (e.g. the axis for delivering the agents), thus providing graphs in a three dimensional plot. For example, it is known that solid tumors are heterogeneous in nature with among other differences, a quiescent inner zone and a proliferative outer zone. An agent may have different effects on the inner zone and the proliferative outer zone. It may be important to target the proliferating zone of solid tumors to assess drugs that target mitosis and mitotic checkpoints and/or pathways which are more active in proliferating zones, for example, C-Met and AKT. Therefore, the methods described herein allow evaluation of therapeutic agents across the entire solid tumor.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Simplified Experimental Systems Used to Evaluate Different Injection Methods

Fluid Dynamics Simulations

Comsol Multiphysics fluid dynamics software was used for simulation. Variables such as flow rate, pore size, pore number, needle length, fluid viscosity, etc. were manipulated to determine effect on fluid deposition outside the needle.

Real Time Visualization of Injections into Gel Slabs

Injection was done in real time and visualized with a Canon EOS Rebel T3i using a Canon EF-S 60 mm Macro Lens. Dyes injected were all standard off the self food coloring (e.g, FD&C Blue No. 1, Brilliant Blue FCF, EU#E133, FD&C Green No. 3, Fast Green FCF, EU#E143, FD&C Red No. 3, Erythrosine, EU#E127)

Gelatin used for injections is commonly known as “ballistics gel” and is designed to simulate animal tissue. Injection conditions were:

Flow rates between 0.70 μL/min and 17 mL/min, vertical retraction rates between 0.5 mm/min and 1 mm/20 sec, as well as no retraction, and injection volumes of 3-5 microliters.

Needle Designs tested included 25 Gauge end port needle from BD Biosciences, 23 Gauge end port needle from BD Biosciences, 26 Gauge porous needles with 5 mm porous region, 26 Gauge porous needles with 3 mm porous region. Other factors varied and/or assessed were the amount of pressure applied by the array onto the top of the gel. Injections were repeated at least 5 times each and visually assessed as to consistency and uniform fluid distribution down the vertical column.

Method 1 was an extrusion method with 25 Gauge end port needle from BD Biosciences. Method 2 was an extrusion method with 26 Gauge porous needle with mm long porous region. Method 3 was a standard injection method with 26 Gauge porous needle with mm long porous region. The extrusion methods were carried out at a fluid flow rate of 0.70 μL/min, a vertical retraction rate of 1 mm/min and 5 microliter injection volume. The standard injection method was a fluid flow rate of 0.70 μL/min and 5 microliter injection volume without vertical retraction.

### Example 2

### Fluorescent Microscopy Images of Three Different Injection Methods

The first two rows were images from extrusion an method with 25 Gauge end port needle from BD Biosciences. The third and forth rows were images from an extrusion method with 26 Gauge porous needle with mm long porous region. The fifth and sixth rows were images from a standard injection method with 26 Gauge porous needle with mm long porous region. The extrusion methods were carried out at a fluid flow rate of 0.70 uL/min, a vertical retraction rate of 1 mm/min and 5 microliter injection volume. The standard injection method was a fluid flow rate of 0.70 uL/min and 5 microliter injection volume without vertical retraction.

### Example 3

### Comparison of Results from Standard Injection Method and Extrusion Method

Results from the standard injection method and extrusion method with respect to efficiency, intratumor signal uniformity and column length were compared. The “novel method” referred herein is the extrusion method. The “standard method” or “previous method” referred herein is the standard injection method.

Experimental Details for “Standard Method”

26 Gauge porous needle with 5 mm long porous region
Flow rate of 0.70 uL/min
No vertical retraction
5 microliter injection volume
Experimental Details for “novel method”
25 Gauge end port needle from BD Biosciences <
Flow rate of 0.70 uL/min
Vertical retraction rate of 1 mm/min
5 microliter injection volume

### Example 4

### Comparison of Average Number of Positive Regions Per Section

This example shows a comparison of the average number of positive regions per section and shows average variance within section of three different injection methods.

Experimental Details for the Extrusion Method

### “Open-POM”=“Extrusion Method”

25 Gauge end port needle from BD Biosciences <
Flow rate of 0.70 uL/min
Vertical retraction rates of 1 mm/min
“PNA-POM”=“Extrusion method with porous needle”
26 Gauge porous needle with 3 mm long porous region
Flow rate of 0.70 uL/min
Vertical retraction rates of 1 mm/min
5 microliter injection volume
“PNA-Standard”=“standard injection method with porous needle”
26 Gauge porous needle with 5 mm long porous region
Flow rate of 0.70 uL/min
No vertical retraction
5 microliter injection volume

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-135. (canceled)

136. A device, comprising a top block comprising a first plurality of holes sized to allow a needle to pass through said top block and a bottom block comprising a second plurality of holes sized to allow a needle to pass through said bottom block, wherein said top and bottom blocks are in a substantially parallel arrangement and wherein said first and second plurality of holes are positioned so as to allow one or more needles to pass through a hole in said top block and said bottom block in a path substantially vertical to a plane of both blocks.

137. A method of operating a device, comprising:
a. providing a device comprising a top block comprising a first plurality of holes sized to allow a needle to pass through said top block and a bottom block comprising a second plurality of holes sized to allow a needle to pass through said bottom block, wherein said top and bottom blocks are in a substantially parallel arrangement and wherein said first and second plurality of holes are positioned so as to allow one or more needles to pass through a hole in said top block and said bottom block in a path substantially vertical to a plane of both blocks
b. inserting one or more needles through said top block and said bottom block into a solid tissue, wherein at least one needle has a control attachment;
c. simultaneously moving said top block away from said bottom block; and
d. injecting at least one agent into said solid tissue.

138. The method of claim 137, further comprising, evaluating an effect of said at least one agent on said solid tissue.

139. A device for delivering an agent to a solid tissue, comprising:
a. two or more needles, wherein said two or more needles comprises a first end and a second end;
b. a reservoir, wherein said reservoir is in fluid communication with said first end of said two or more needles;
c. a plunger, wherein said plunger is coupled to said reservoir; and
d. a controller, wherein said controller controls a rate of fluid delivery when said two or more needles are withdrawn from said solid tissue.

140. A method of delivering an agent to a solid tissue of a subject, comprising:
a. inserting a plurality of needles into said solid tissue; and
b. delivering said agent to said solid tissue, wherein a rate of delivery of said agent is substantially controlled by a rate of needle withdrawal from said solid tissue.

141. The method of claim 140, wherein said needle is part of a needle array device.

142. The method of claim 141, wherein said needle array device comprises 5 or more needles.

143. The method of claim 142, wherein each of said needles comprises a different agent.

144. The method of claim 142, wherein at least two of said needles comprises a same agent at different concentrations.

145. The method of claim 140, wherein said agent comprises an anti-cancer agent.

146. The method of claim 145, wherein said anti-cancer agent comprises a small molecular agent.

147. The method of claim 140, wherein said agent comprises a position marker.

148. The method of claim 140, wherein said agent comprises a negative control.

149. The method of claim 140, wherein said agent comprises a positive control.

150. The method of claim 140, wherein said agent is present in said solid tissue at a therapeutically effective concentration.

151. The method of claim 140, wherein said agent is delivered to said solid tissue in an amount that is undetectable outside said solid tissue.

152. The method of claim 140, wherein said agent is present in said solid tissue at a therapeutically effective concentration, and wherein outside said solid tissue said agent is present at a concentration that is undetectable.

153. The method of claim 140, wherein said agent is undetectable outside said solid tissue.

154. The method of claim 140, wherein said solid tissue comprises a tumor.

155. The method of claim 154, wherein said tumor is selected from the group consisting of: a primary tumor, an invasive tumor and a metastatic tumor.

156. The method of claim 140, wherein said solid tissue is selected from the group consisting of: brain, liver, lung, kidney, prostate, ovary, spleen, lymph node, thyroid, pancreas, heart, skeletal muscle, intestine, larynx, esophagus, skin, and stomach.

157. The method of claim 140, further comprising evaluating an effect of said agent on said solid tissue.

158. The method of claim 157, wherein said evaluating is performed in vitro.

159. The method of claim 157, wherein said evaluating is performed in vivo.

160. The method of claim 157, wherein said evaluating comprises histology sectioning.

161. The method of claim 157, wherein said evaluating comprises imaging said solid tissue.

162. The method of claim 161, wherein said imaging is selected from the group consisting of: radiographic imaging, magnetic resonance imaging, positron emission tomogoraphy, and biophotonic imaging, or any combination thereof.

163. The method of claim 161, wherein said imaging occurs at a time point selected from the group consisting of: before introduction of said agents, during introduction of said agents, and after introduction of said agents, or any combination thereof.

164. The method of claim 157, wherein said evaluating comprises analyzing at least one biomarker for biological activity.

165. The method of claim 164, wherein said biological activity is selected from the group consisting of: tumor cell death, cell signal changes, proliferation changes, and mitotic changes, or any combination thereof.

166. The method of claim 157, wherein said evaluating comprises detecting an effect of said one or more agents on a proliferative gradient of said solid tissue.

167. The method of claim 157, wherein said evaluating comprises detecting an effect of said one or more agents on multiple microenvironments of said solid tissue.
